(19) United States
(12) Patent Application Publication
(10) Pub. No.: US 2008/0050347 A1
(43) Pub. Date: Feb. 28, 2008

(54) STEM CELL THERAPY FOR CARDIAC VALVULAR DYSFUNCTION

(76) Inventor: Thomas E. Ichim, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/844,305

(22) Filed: Aug. 23, 2007

Related U.S. Application Data
(60) Provisional application No. 60/823,363, filed on Aug. 23, 2006.

(51) Int. Cl. A61K 35/12; A61F 2/02; A61P 9/00

(52) U.S. Cl. 424/93.7; 604/522; 623/23.72

(57) ABSTRACT

Disclosed are methods, compounds and compositions useful for treatment of a patient with valvular dysfunction. The invention relates to using stem cells, modified stem cells, derivatives thereof, and agents stimulatory to stem cells in order to substantially ameliorate, and in some cases induce a therapeutic benefit, to a patient suffering from a dysfunction of the mitral, aortic, tricuspid, or pulmonary valve. In some embodiments the invention treats the valve dysfunction itself, whereas in other embodiments treatment of associated cardiac structures is performed. Furthermore, in other embodiments the invention permits physiological compensation for the valve dysfunction, prolonging the time until surgical intervention is needed.

### CROSS REFERENCE TO RELATED APPLICATIONS

This application claims priority to U.S. Provisional Application No. 60/823,363, filed on Aug. 23, 2006, entitled STEM CELL THERAPY FOR CARDIAC VALVULAR DYSFUNCTION, the entire disclosure of which is incorporated herein by reference in its entirety.

### FIELD OF THE INVENTION

The invention disclosed relates in general to the field of cardiology. More specifically, it relates to methods, cells, and compositions of matter useful for treatment of valvular dysfunction. More specifically, the invention relates to cellular therapies, methods of enhancing activity of cellular therapies, and means of manipulating endogenous cells to effectuate restoration of valvular sufficiency, and/or enhance ability of the heart to compensate for valvular insufficiency in order to delay need for surgical intervention, and/or accelerate cardiac competency following surgical or other interventions for valvular insufficiency.

### BACKGROUND OF THE INVENTION

At present, one of the leading causes of death in the Western world is cardiovascular disease, which causes an estimated annual death toll of more than ten million people. These diseases, for example chronic hypertension, left ventricular hypertrophy, and myocardial ischemia often culminate in heart failure. The current invention deals with one of the instigators of heart disease called valvular dysfunction. In some situations valvular degeneration leads to other types of heart disease and eventually to heart failure, whereas in other situations, various heart diseases precipitate valvular dysfunction. Ischemic heart disease is in some cases, for example, a causative agent in mitral valve dysfunction (1).

The human cardiac system has four valves whose function is ensuring unilateral flow of incoming blood to the systemic circulation as it is being pumped through the heart. In the left part of the heart, separating the left atrium and left ventricle is the mitral valve (or otherwise termed the “bicuspid” valve). The mitral valve is the only valve that normally consists of two flaps, hence the alternative name bicuspid. The aortic valve separates the left ventricle from the ascending aorta. The aortic valve opens during systole in order to allow blood to exit the heart and enter systemic circulation. On the right hand side of the heart is the tricuspid valve, which separates the right atrium from the right ventricle, and as the name implies it is comprised of three flaps, or cusps. Also on the right side of the heart resides the pulmonary valve, which separates the right ventricle from the pulmonary artery, it acts as the gatekeeper to prevent blood from the lungs to reflux back into the heart. In contrast to the mitral and tricuspid valves, which are considered “arterioventricular valves”, the pulmonary and aortic valves are considered “semilunar valves” since both of these valves are composed of 3 semilunar shaped cusps. All valves are held in appropriate position by various structures. Degeneration of said structure, leads in some situations to valvular deterioration, whereas in other situations malfunctioning valves precipitate degeneration of said associated structures. Relevant cardiac structures include the aortic root, the annulus, the papillary muscles, the cordae tendinaea, and the heart wall. The cordae are bands of fibrous tissue which attach on one end to the edges of the tricuspid and mitral valves of the heart and on the other end to the papillary muscles, thus serving to anchor the valves. The aortic root maintains the three dimensional structure of the normally tricuspid aortic valves in such a manner that during closing the valve cusps are properly aligned and blood does not regurgitate into the ventricle during diastole.

Dysfunction of the cardiac valves can be associated with congenital abnormalities (2), acquired degeneration (3, 4), or a mixture of the two in which a genetically acquired trait leads to increased propensity towards a degenerative state (5). Severity of symptoms and need for surgical or other intervention is dependent on a variety of factors intrinsic to the state of degeneration, as well as the affected valve. Commonly, valve abnormalities can be divided into ones that result in the backflow of blood due to inability to properly close (regurgitation), or alternatively, when valves do not properly open the condition is called stenosis. Below are reviewed some of the conditions associated with valvular degeneration:

Mitral valve prolapse is condition of the heart valve characterized by the displacement of an abnormally thickened mitral valve leaflet into the left atrium during systole (6). Specifically, prolapse is considered to be occurring when the mitral valve leaflets are displaced more than 2 mm above the high point of the mitral annulus (7). There are two forms of mitral valve prolapse: The nonclassic form, which carries a low risk of complications; and the classical form, which when severe, leads to mitral regurgitation, infective endocarditis, and occasionally sudden cardiac death. Nonclassical mitral prolapse is usually defined by mitral leaflets having a thickness of less than 5 mm, whereas the classical variant has a thickness of greater than 5 mm. Classical prolapse is further subdivided into symmetric and asymmetric. In the symmetrical variant the leaflet tips meet each other, or coapt, on a shared region on the annulus. This is in contrast to the asymmetric variant in which one leaflet is closer to the atrium during coaption as compared to the other leaflet. The term flail and non-flail refers to whether the leaflet tip becomes concave toward the left atrium. In some cases the flailing is associated with weakness or rupture of the supporting chordiae tendinae. Each year there are approximately half a million diagnosis of mitral valve disease in the US alone (8). However the vast majority of these are classified by echocardiogram as not needing surgical intervention due to low regurtitant volume. There is controversy regarding the extent of treatment that should be implemented during mitral regurgitation that is asymptomatic (9). The overall consensus seems to be that due to the invasiveness of surgery associated with either mitral valve replacement or repair, the criteria for intervention should remain at the current recommended values for left ventricle size, left ventricle function, and pulmonary hypertension (10).

Mitral valve insufficiency (regurgitation) is one of the symptoms of mitral valve prolapse, however there are a wide variety of causes in addition to prolapse that may cause regurgitation. Other causes include: coronary artery disease (ischemic regurgitation, post myocardial infarct ischemia, myxomatous degeneration of the mitral valve, heart trauma, Marfan's syndrome, rheumatic heart disease, and administration of certain medications. Mitral valve insufficiency is classified into primary and secondary (11). The main cause of primary mitral insufficiency in the US is myxomatous degradation of the valve. Myxomatous refers to a degeneration of the connective tissue, especially collagen. Interstitial cells in myxomatous valves have features of activated myofibroblasts and express excessive levels of catabolic enzymes, causing pathological remodeling through overactivation of matrix metalloproteases without synthesis of new extracellular matrix (11). This type of degeneration causes malcoaption of the valve leaflets due to either stretching out of said leaflets, and/or elongation of the chordae tendineae. The major cause of secondary mitral insufficiency is dilation (or deformation) of the left ventricle as a result of ischemic heart disease, cardiomyopathy (including non-ischemia associated cardiomyopathy), and aortic insufficiency. This causes alteration in the overall cardiac structures so as to result in valve misalignment (malcoaption) during closing. Mitral insufficiency, whether caused by mitral valve prolapse, or other causes, results in accumulated volume in the right atrium (12). This volume overload generally leads to a type of myocardial hypertrophy called “eccentric hypertrophy” since the chamber radius increases in size and the sarcomeres start multiplying in series (13). During the compensated phase, the eccentric hypertrophy of the myocardium allows maintenance of normal stroke volume. However, during the decompensated phase, heart failure occurs. Some manifestations of mitral regurgitation occur as an acute response, which usually occurs with a spontaneous chordae tendineae rupture secondary to myocardial infarction, or traumatic injury (14). This causes a sudden volume overload in the left atrium, as well as ventricle. The volume overload on the left ventricle increases amount of energy needed for the left ventricle to pump out the blood. On the other hand, the regurgitation causes build-up of volume in the left atrium during systole, which results in increased pressure on the lungs and is manifested through acute pulmonary edema and dyspnea. In some cases, cardiac mechanism can resist the acute phase and begin compensating as described previously. Current non-surgical treatments involve preload and afterload reduction (15, 16). One common treatment is administration of furosemide (Lasix), which acts as a preload reducer by increasing excretion of water through inhibiting sodium and chloride reabsorption in ascending loop of Henle and distal renal tubule. Another commonly used treatment is nitroglycerin, which in addition to being a preload reducer, also causes anti-anginal activity. Afterload reducers such as captopril are also used in some patients. Anti-arrythmics such as digoxin are also used in some patients to prevent atrial fibrillations. Anticoagulants such as warfarin are also used to prophylactically avoid formation of thrombus. Surgical intervention is recommended when the patient becomes severely symptomatic (NHYA class III or IV) and presents with significant reduction of left ventricular function (10). If the mitral annulus is dilated, an annuloplasty may be successful in alleviating the degree of regurgitation (annuloplasty involved insertion of a ring around the annulus. Another option is surgical shortening of the chordae and/or papillary muscles. Complete replacement of the mitral valve replacement is usually the final option. A variety of valves are currently available including completely artificial, xenogenic, and hybrids thereof.

Mitral valve stenosis (narrowing) occurs when the mitral valve does not fully open, usually as a result of mitral valve leaflets that are thickened, commissures that are fused, and/or chordae tendineae that are thickened and shortened. The incomplete opening causes built-up of pressure in the right atrium. This pressure built-up causes concentric myocardial hypertrophy in that the radius of the chamber remains the same, but the surrounding myocardium thickens through the addition of sarcomeres in parallel. In a similar manner to eccentric myocardial hypertrophy, this type of hypertrophy acts as a mechanism of compensation, and in the long-term results in heart failure. On average approximately 40% of patients with mitral stenosis have some degree of atrial fibrillation (17). Specifically, mitral stenosis patients with a lower cardiac index, and smaller mitral valve area appear to be more susceptible to atrial fibrillation (18). Mitral stenosis is usually caused as a result of rheumatic heart disease, by calcification of the mitral valves, or due to various congenital abnormalities (19). Medical therapy of mitral stenosis usually aims at preventing arrhythmias that are associated with this condition. Commonly used drugs include digoxin. Symptomatic mitral regurgitation in patients with moderate-to-severe stenosis (mitral valve is <1.5 cm2), and/or pulmonary hypertension >50 mmHg is treated usually by balloon valvotomy. Successful balloon valvotomy can actually double the average area of the valve in order to cause a 50-60% decrease in the transmitral gradient. This usually induces reduction of symptoms. Other treatment means for advanced patients includes surgical commissurotomy, percutaneous mitral valvotomy or mitral valve replacement. It is to be noted, however, that the main intervention used clinically is repair of the mitral valve and not replacement

Aortic insufficiency (AI), also known as aortic regurgitation, or aortic valve prolapse is characterized by weakening or malformation of the aortic valve preventing it from fully closing during ventricular distole (20). In some patients abnormalities of the proximal aortic root may also be causal. This causes retrograde blood flow from the aorta into the left ventricle leading to volume overload in the left ventricle due to the need to eject the regurgitant volume from the ventricle in addition to the usual left ventricle volume. Eventually, the volume overload leads to eccentric hypertrophy of the left ventricle, as in the case of the mitral insufficiency. In past half century, rheumatic fever was the primary cause of aortic insufficiency. Now that antibiotics are used to treat rheumatic fever, other causes are being seen with increasing frequency. These include congenital conditions (abnormalities of the valve which are present at birth), endocarditis (valve infection), high blood pressure, Marfan's syndrome, aortic dissection (a tear in the lining of the aorta), ankylosing spondylitis, Reiter's syndrome, syphilis (now rare), and other disorders. The most common congenital abnormality causing aortic insufficiency is a bicuspid aortic valve (21). The aortic valve normally has three cusps, but a bicuspid aortic valve has only two. It may, therefore, not open or close completely due to altered geometry. Bicuspid aortic valve is a common abnormality and occurs in 1-2% of people. This is the second most common cause of aortic valve disease requiring surgery. Such valves may function normally for years before becoming stenotic. Patients with a bicuspid aortic valve usually require antibiotic prophylaxis before dental procedures but generally no other special precautions are required other than cardiological examination. In addition to chronic AI, acute AI presents with a different subset of symptoms since it lacks the hypertrophied myocardium to compensate for increased volume load. Acute is often caused by endocarditis or sudden trauma. Volume overload in the left ventricle results in an early closure of the mitral valve during diastole as a natural defense mechanism to protect the pulmonary venous system from the high-pressure regurgitant backflow coming from the high-pressure LV chamber.

The most widely used therapy for chronic, asymptomatic AI is vasodilator therapy and observation, or “watchful waiting”. Vasodilators reduce systemic vascular resistance, allowing more forward flow to occur therefore improving cardiac output. Catecholamines by virtue of increasing peripheral vascular resistance are detrimental to the course of AI. Therefore, oral hydralazine by reducing end-diastolic volume and increasing ejection fraction has been found to be beneficial in certain patient populations with AI. Calcium channel blockers such as nifedipine also are beneficial by evoking a rapid reduction in peripheral vascular resistance. Alpha-blockers such as prazosin or direct vasodilators like nitroprusside are also useful medical therapy for AI. Patients which develop symptoms of congestive heart failure, systolic diameter greater then 55 mm, diastolic diameter greater then 75 mm, and/or left ventricular ejection fraction less then 55% are recommended to undergo surgical intervention (10).

Surgical treatment is specific to the patient conditions. In some cases, correction of dilated aortic root is performed with annuloplasty. In other cases correction of aneurysmal dilatation of the ascending aorta is required and performed by excision, graft placement and coronary artery reimplantation. When AI is caused by perforated valve leaflet, a pericardial patch is placed surgically. In other cases, artificial, xenogenic, or hybrids thereof are introduced. One specific type of surgery commonly used is the Ross procedure, whereby the defective aortic valve is replaced with an autotransplant of the patient's pulmonary valve. The original pulmonary valve is replaced with a prosthetic. Since the pulmonary valve is less hemodynamically needed, its replacement is by prosthesis does not cause as much deterioration of the cardiac structures as would a similar replacement of a more important valve such as the aortic or mitral valve. It is to be noted that unlike the mitral valve, which can often be repaired surgically, the aortic valve usually requires replacement.

Aortic valve stenosis, is caused by narrowing of the aortic valve of the hearts, leading to built-up of pressure in the left ventricle, as in the case of the mitral valve stenosis, where pressure builds up in the atrium and leads to concentric hypertrophy. As in mitral valve stenosis, this pressure built-up causes concentric myocardial hypertrophy in that the radius of the chamber remains the same, but the surrounding myocardium thickens through the addition of sarcomeres in parallel. In a similar manner to eccentric myocardial hypertrophy, this type of hypertrophy acts as a mechanism of compensation, and in the long term results in heart failure. Also similar to mitral stenosis, aortic stenosis is usually caused as a result of rheumatic heart disease, by calcification of the aortic valves, or due to various congenital abnormalities. The most common cause of aortic valve disease requiring surgery is called “senile aortic calcification,” When a valve becomes worn, the calcium deposition occurs directly, as well as accumulation of osteoblastic cells is seen. The calcium deposit restricts the motion of the valve leaflets. This in many cases prevents the valve from properly opening. Treatment for aortic valve stenosis is usually aortic balloon valvotomy, or valvuloplasty. Said treatments are usually recommended when patients become symptomatic, displaying signs of congestive heart failure or syncope. Additionally, treatment is initiated when the valve area becomes less than 0.6 cm2, and/or significant reduction in left ventricular ejection fraction is observed.

Tricuspid valve insufficiency occurs when the tricuspid valve (located between the right atrium and right ventricle) does not close tightly enough (malcoaption) to prevent leakage, and as a result a portion of blood moves in a retrograde direction back into the right atrium. This condition is also called tricuspid valve regurgitation and tricuspid incompetence. Primary causes of tricuspid insufficiency include endocardial cushion defects, blunt trauma or after mitral valve surgery, Ebstein's anomaly (i.e., downward displacement of a distorted tricuspid cusp into the right ventricle), and carcinoid syndrome. Secondary causes include dilation of the right ventricle, pulmonary hypertension, and/or pulmonary valve stenosis. Infective endocarditis, cor pulmonale, post-infarction remodeling, and certain drugs such as fenfluramine also are associated with tricuspid insufficiency. Medical treatment includes diuretics such as lasix in order to inhibit fluid overload, digoxin to control atrial fibrillation and increase contractility, ACE inhibitors such as captopril to induce afterload reduction, and anticoagulants to inhibit thrombus formation. In advanced cases surgical remodeling of the tricuspid annulus is performed. The use of remodeling is recommended when the tricuspid valve area is <1.5 cm2.

Tricuspid valve stenosis occurs when blood flow is obstructed out of the right atrium. This is similar to mitral stenosis with the exception that the changes occur in the right atrium as opposed to the left. Concentric hypertrophy ensues, and the causative factors are similar to mitral stenosis mentioned above. However, tricuspid stenosis is usually much rarer than mitral stenosis. Medical treatment includes administration of digoxin to strengthen myocardial systolic contractions, as well as to upregulate carotid sinus nerve activity, and anticoagulants to prevent thrombosis formation. Tricuspid stenosis treatment by surgical is performed when right heart failure is detected, or deemed pending. With tricuspid valve replacement, the risk of thrombosis is significant and many surgeons advise warfarin therapy for either artificial, xenogenic, or hybrid valves. Percutaneous balloon valvuloplasty has been used successfully, as long as concomitant regurgitation is not significant.

Pulmonic stenosis is a narrowing of the semilunar pulmonary valve, or associated structures that are situated between the lungs and right ventricle. Pulmonary stenosis may be valvar, supravalvar, or subvalvar (infundibular or mid cavity, ie, double-chamber right ventricle); it may also be in the branch pulmonary arteries. Such stenosis usually originates primarily as a congenital abnormal abnormality, most commonly associated with Teratology of Fallot. Early stage pulmonary stenosis is usually silent and difficult to diagnose. In advanced states a short ejection murmur is heard in the area of the pulmonic valve, classically with a “click” which signifies the walls of the pulmonic valve snapping outward with each pumping stroke. Patients with trivial (gradient<25 mm Hg) or mild (gradient<50 mm Hg) pulmonary stenosis are usually observed clinically at a yearly basis, but more frequently during infancy and adulthood. Patients with moderate (gradient 50-79 mm Hg) and severe (gradient>80 mm Hg) obstruction require surgical or percutaneous intervention, which includes balloon pulmonary valvuloplasty, surgical remodeling, or balloon atrial septostomy. It is important to note that after balloon pulmonary valvuloplasty approximately 25% patients are rehospitalized to address related complications and restenosis.

Pulmonic insufficiency is characterized by retrograde flow from the pulmonary artery into the right ventricle during diastole. Low-level pulmonic insufficiency is present in nearly all individuals, and incidence and severity increases with age. In advanced pulmonic insufficiency, significant regurgitation may lead to impairment of right ventricular function, right-sided volume overload and heart failure. Often, pulmonic regurgitation is not the primary process but occurs secondary to an underlying process such as pulmonary hypertension or dilated cardiomyopathy. Pulmonic regurgitation is seldom severe enough to require surgical or even medical treatment because the right ventricle normally adapts to low-pressure volume overload without difficulty. In situations where such adaptation occurs pathologically, volume overload leads to right-sided heart strain and, ultimately, heart failure. Prior to this, surgical interventions that are used include surgical reconstruction or replacement of the pulmonic valve with an artificial, xenogenic, or hybrid valve.

As can be seen from the above description, pathology can be associated with one or more of the four cardiac valves. The cause of the pathology can be either the valve itself degenerating, the supporting structures of the valve degenerating, or the cardiac ventricle changing shape so as to not allow proper closure of the specific valves during the appropriate phase of the cardiac cycle. The degeneration of some valves is usually more detrimental to the patient than other valves. For example mitral and aortic valvular degeneration is usually more harmful than degeneration of the tricuspid or pulmonary valve.

In numerous cases of valvular diseases, the main treatment, at least in the asymptomatic or early phase, is “watchful waiting”. Depending on the specific valve(s) affected and underlying patient characteristics, the various trigger points to begin medical or surgical interventions vary.

For example, in mitral regurgitation, Rosenhek et al (22) followed a group of 132 consecutive enrolled asymptomatic patients (age 55±15 years, 49 female) with severe degenerative mitral regurgitation (flail leaflet or valve prolapse) for 62±26 months.

Specific patient characteristics that allowed inclusion in this prospective study were: a) severe mitral of degenerative origin documented by echocardiography (prolapse, flail leaflet), lack of symptoms, normal LV function (fractional shortening≧0.32, ejection fraction≧0.60, end-systolic diameter<45 mm, or end-systolic diameter index<26 mm/m2, considering body surface area), Doppler sonographically estimated systolic pulmonary artery pressure<51 mm Hg, and sinus rhythm. No patients with additional hemodynamically significant valve lesions (more than mild), nor patients with congenital heart disease, ischemic heart disease, or cardiomyopathy were included. For the purpose of the study, mitral valve prolapse was defined as displacement of 1 or both of the leaflets into the left atrium below the mitral annulus level during systole. Flail leaflet was classified as when the leaflet tip turned outward, becoming concave toward the left atrium. Quantification of the extent of mitral regurgitation was performed by an integrated approach that included valve morphology, left ventricle volume load, width of the proximal regurgitant jet, proximal flow convergence, and pulmonary venous flow pattern. Severe mitral regurgitation was defined as: A flail leaflet with clearly visible coaptation defect. In patients with prolapse without flail, a proximal jet width>6 mm and a flow convergence radius>7 mm at a Nyquist limit of 55 to 65 cm/s was considered severe mitral regurgitation. Left ventricle enlargement with normal function in the absence of any causes of LV dilatation other than mitral regurgitation was considered supportive of severe mitral regurgitation. Surgical intervention was performed when patients experienced onset of symptoms or if an asymptomatic patient developed 1 or more of the following: a) LV end-systolic diameter≧45 mm or end-systolic diameter index 26 mm/m2; b) fractional shortening<0.32; c) ejection fraction<0.60; d) systolic pulmonary artery pressure>50 mm Hg; or e) recurrent atrial fibrillation. During the follow up period 8 patients died and 32 had developed criteria for surgery. Based on these data, the authors of the study suggested that “prophylactic surgery for all patients with asymptomatic severe degenerative MR can definitely not be recommended”.

The purpose of medical intervention in valvular diseases is to prolong the time needed before the patient undergoes surgery. Specific interventions also seek to advert consequences of the specific valve defect. For example, valvular defects always result in altered hemodynamics of the blood, which causes an increased propensity for clot formation. Prophylactic anticoagulant therapy has been demonstrated useful in patients with mitral stenosis, and mitral stenosis-associated atrial fibrillation (23). Certain patient populations are more likely to suffer from hemodynamically-induced clotting, and these benefit also from treatment with anticoagulants (24). In general the current practice is to ensure proper anticoagulation in patients with a variety of valvular diseases (25). Additional medical care includes the use of digoxin to prevent atrial fibrillation (26), as well as to enhance contractile function (27). Diuretics are used in patients with valvular dysfunction that are associated with volume overload, in order to decrease preload (28). ACE inhibitors and other vasoactive drugs such as nitroprusside, hydralazine, and nifedipine are used in some valvular diseases for decreasing afterload pressure (29). Overall, medical intervention in valvular diseases only inhibits progression, although to a minor extent.

Surgical approaches to valvular degeneration offer cure, however various drawbacks exist. These include the need for open-heart surgery, which carriers significant morbidity and mortality, as well as the fact that a substantial number of patients are ineligible for surgery due to other underlying diseases. Common surgical interventions include annuloplasty, valvoplasty, valve repair, and valve replacement. A wide variety of artificial and semiartificial valves have been developed for specific valves that require replacements. Particularly prominent is the St Jude's valve for aortic insufficiency. Another problem with surgical approaches is the limited half-life of certain artificial valves, limited re-endothelialization of said valves, as well as development of restenosis. Particularly, thrombotic complications are known to occur after a mechanical valve implantation, therefore, lifelong anticoagulation is essential. Even with anticoagulation, the patient may still experience bleeding or other thrombotic complications. On the other hand, tissue valves (allogeneic or xenogeneic) are generally free of such complications, and patients do not require anticoagulation in the long-term. However, as stated above, tissue valves have relatively limited durability compared to mechanical valves. As a result these patients require future reoperation.

Due to the dangers of open-heart surgery, much effort is being directed towards percutaneous valvular repair. On such percutaneous means involves a trans-septal delivery of a clip device to grasp the mitral leaflet edges and create a double orifice to allow for increased closing of the valve.

Feldman et al (30) selected 27 patients for percutaneous mitral repair using the following criteria for mitral regurgitation: Patients with moderate-to-severe or severe MR who were symptomatic or asymptomatic patients with moderate-to-severe or severe MR with compromised left ventricular function (LVEF<60% or left ventricular end-systolic dimension>45 mm) were selected. Moderate-to-severe, or severe mitral regurgitation was defined according to the ACC/AHA Joint Task Force Recommendations for treatment of valvular heart diseases (10). Specifically, patients must have a minimum of three of the following criteria, one of which must be quantitative (i.e., 4, 5, or 6): 1. Color flow jet may be central and large (>6 cm2 or >30% of left atrial area) or smaller if eccentric, encircling the left atrium; 2. Pulmonary vein flow may show systolic blunting or systolic flow reversal; 3. Vena contracta width>0.3 cm measured in the parasternal long-axis view; 4. Regurgitant volume of >45 ml/beat; 5. Regurgitant fraction>40%; and 6. Regurgitant orifice area>0.30 cm2. Using echocardiographic guidance, 24 patients had successful clip implantation. Clips were successful implanted in 24 patients with no procedural complications, however four major 30-day adverse events were reported. After six months, 13 patients had ≦2+ mitral regurgitation. Patient who had subsequent surgery had elective mitral valve repair or replacement. This study demonstrated that percutaneous mitral valve repair can be performed with relative safety and that mitral regurgitation can be reduced significantly without compromising surgical options. Unfortunately, this trial was a Phase I, first in man, investigation using small patient sample size and short term follow-up.

Webb et al (31) successful implanted 14 patients suffering from advance aortic stenosis with an expandable trileaflet equine valve prosthesis using a percutaneous, femoral artery-to-aorta approach. The patients treated were deemed ineligible for conventional intervention due to operative risk. While there were no intraprocedural deaths, at 30 follow-up 2 deaths were noted: one the result of iliac perforation, and the second associated with left coronary obstruction by a displaced native aortic valve leaflet. Due to the experimental nature of this intervention, as well as the short follow-up, numerous advances have to be made before percutaneous valve implantation becomes a widely-applicable advancement in this field.

Webb et al (32) performed percutaneous annuloplasty in 5 patients with post myocardial infarction mitral regurgitation who had a functional mitral regurgitation score of 2+ on a scale of 4+. Exclusion criteria included left ventricular ejection fraction<30%, structural mitral valve disease, mitral annular calcification, prior endocarditis or mitral surgery, creatinine>2.0 mg/dL, coronary sinus pacing leads, anticipated revascularization, inability to take aspirin or clopidogrel, or a life expectancy<1 year. Insertion of a Viking annuplasty device was performed, resulting in the mitral regurgitation score decreasing on average from 3.0±0.7 to 1.6±1.1 at the last postimplantation visit when the device was intact.

Khambadkone et al (33) reported a series of 59 patients that received pulmonary valve implants via the percutaneous route. The right ventricular (RV) pressure (64.4±17.2 to 50.4±14 mm Hg), right ventricular outflow tract gradient (33±24.6 to 19.5±15.3, and pulmonary regurgitation (grade 2 or greater before, none greater than grade 2 after, P<0.001) decreased significantly after percutaneous pulmonary valve implantation. MRI showed significant reduction in the pulmonary regurgitation fraction (21±13% versus 3±4%) and in right ventricular end-diastolic volume (94±28 versus 82±24 mL·beat-1·m-2, P<0.001) in 28 of the patients (age 19±8 years) at the follow up average of 9.8 months post procedure.

### SUMMARY OF THE INVENTION

Numerous pathological situations occur as a result of valvular degeneration and abnormalities. In many cases, the valvular abnormality is detected at a stage where pathology is not initiated and the patient has the option of either “watchful waiting” or taking various medical therapies. When the valvular dysfunction or abnormality causes begins to initiate pathological processes to a certain extent, then surgical intervention is necessary. One aspect of the current invention is to provide cells, methods of treatment, and various compounds, that can be used for preventing deterioration of the valvular and non-valvular cardiac structures so as to delay the need for surgical intervention. The impetus for delaying said surgical intervention is because of certain risks associated with surgery, as well as the inability of certain subsets of patients to undergo surgery due to other underlying medical conditions. In some aspects the methods disclosed herein further can include the step of identifying a patient in need of therapy for valvular dysfunction or identifying a patient with a particular heart condition or disorder, in particular, a valvular condition or condition caused by a valvular condition. Examples of some conditions are described herein. The identified patient can be treated according to the methods described herein.

The various cellular, anatomical and hemodynamic components that dictate the need for surgery are numerous. The invention teaches various means for addressing either singularly, or in plurality, the various components associated with the need for surgery so as to allow for regeneration, or non-pathological compensation of said structures.

For example, valvular degeneration or abnormality cause in some situations regurgitation of blood into either cardiac ventricle or atria during specific phases of the cardiac cycle, said regurgitation leads to reduced cardiac output, as well as volume overload in the specific ventricle or atria. For example, a regurgitant aortic valve will cause volume overload of the left ventricle, whereas a regurgitant mitral valve will cause volume overload of the right atrium. Said volume overload leads to eccentric hypertrophy of the ventricle surrounding the area of overload, and said hypertrophy eventually results in heart failure or sudden death. On the other hand, obstruction of valves, such as aortic or mitral valve stenosis, leads to pressure overload in the left ventricle or left atrium, respectively. Said pressure overload leads to myocardial concentric hypertrophy, which is also associated with heart failure and sudden death. Said concentric hypertrophy, amongst other things, decreases the amount of oxygen available to the myocardium (due to larger muscle volume), as well as decreased contractility (causing decreased cardiac output). It is known that subsequent to valve repair or replacement, myocardial hypertrophy, both concentric and eccentric are reversible (34, 35). Given that the actual pathology of valvular diseases can be reversible, one aspect of the invention is to utilize both endogenous and exogenous means to induce reversibility of pathology associated with heart failure. It is known that in addition to ventricular remodeling, cardiac valves are also capable of remodeling themselves. Accordingly, one aspect of the current invention teaches means of inducing a repair program in said affected cardiac valves so as to cause beneficial remodeling and restore normal function, without the need for surgical intervention.

Given that the lag time between identification of a valvular disease or abnormality, and the need for surgery can be years, the invention teaches a variety of means for inducing regeneration, either of the valves or the associated cardiac structures, in a spectrum of time. In some aspects the need for inducing regeneration may be more immediate, as in a patient with severe valvular disease who is on the threshold for surgical intervention. In these cases the invention provides solutions that are relatively rapid acting. In other cases, the need for regeneration may not be so immediate, for these situations, means are provided that allow for a relatively protracted regenerative process.

The invention discloses not only methods and means of stimulating regeneration prior to surgical intervention, but also in conjunction with surgery, or post surgery to enhance beneficial aspects of said surgery. In one aspect of the invention, means are thought for accelerating endothelialization of surgically, or percutaneous, implanted prosthetic devices, be they purely artificial, xenogenic, allogeneic, or a hybrid. In another aspect of the invention, means are provided for increasing the physiological integration of implanted devices into the heart. In another aspect of the invention, means are thought for accelerating the healing process subsequent to surgical, percutaneous, or other types of cardiac intervention. In another aspect of the invention, means are thought for assisting cardiac structures to perform optimally subsequent to implantation of devices that assist valvular function. In another aspect of the invention, means are thought for inhibiting pathological processes that arise from implantation of a device for increasing valvular function, surgical procedures for enhance valvular function, or other repair/modification procedures associated with valvular function.

One aspect of the invention is a method of treating valvular heart disease comprising administration of cells, and/or products of said cells, and/or cell stimulatory compounds, and/or cardiac protective factors to an individual in need thereof. Said valvular heart disease may be a state in which suboptimal function of one or more of the following cardiac valves is present: aortic valve, pulmonary valve, tricuspid valve, and mitral valve. Said suboptimal function includes inability to properly close at the time when complete closing is physiological, said reason for improper closure is selected from a group consisting of: malcoaptation, perforation of said valve, and altered mechanical properties. The inability to properly close may result in regurgitation.

The valvular heart disease may be, for example, aortic regurgitation or may be mitral regurgitation, and such aortic and/or mitral regurgitation may be due to an abnormality of the leaflets. The abnormality of the leaflets may be due to for example, a congenital abnormality, artherosclerotic degeneration, ineffective endocarditis, rheumatic heart disease, connective tissue disease, antiphospholipid syndrome, or the use of drugs. The leaflet abnormality-inducing drug may be an anti-anorectic drug, for example selected from the group consisting of fenfluramine, fenfluramine-phentermine, dexfenfluramine, and an ergotamine. The abnormality of the leaflets may be due to for example, trauma or hemodynamic jet lesion. The aortic or mitral regurgitation may be due to an abnormality of the aortic annulus and/or aortic root that is for example, idiopathic, congenital, aortic root dilation, associated with a connective tissue disease, aoroannular ectasia, associated with Marfan syndrome, associated with Ehlers-Danlos syndrome, associated with osteogenesis imperfecta, aortic dissection, or associated with syphilitic aortitis.

In one aspect of the invention said cells are selected from a group consisting of stem cells, committed progenitor cells, and differentiated cells. In a further aspect, said stem cells are selected from a group consisting of: embryonic stem cells, cord blood stem cells, placental stem cells, bone marrow stem cells, amniotic fluid stem cells, neuronal stem cells, circulating peripheral blood stem cells, mesenchymal stem cells, germinal stem cells, adipose tissue derived stem cells, exfoliated teeth derived stem cells, hair follicle stem cells, dermal stem cells, parthenogenically derived stem cells, reprogrammed stem cells and side population stem cells. Selection of cells to be used in the practice of the invention is performed based on a number of relevant factors to the clinical utilization, including patient characteristics, availability of said cells, and need for immune suppression or other interventions when cells are administered.

One aspect of the invention involves treatment of valvular degeneration through administration of totipotent embryonic stem cells, said totipotent embryonic stem cells expressing one or more antigens selected from a group consisting of: stage-specific embryonic antigens (SSEA) 3, SSEA 4, Tra-1-60 and Tra-1-81, Oct-3/4, Cripto, gastrin-releasing peptide (GRP) receptor, podocalyxin-like protein (PODXL), Rex-1, GCTM-2, Nanog, and human telomerase reverse transcriptase (hTERT).

In another aspect of the invention, patients with valvular degeneration are treated with a therapeutically effective amount of cord blood stem cells, said cord blood stem cells may be identified by expression of markers selected from a group comprising: SSEA-3, SSEA-4, CD9, CD34, c-kit, OCT-4, Nanog, CD133 and CXCR-4, and lack of expression of markers selected from a group consisting of: CD3, CD45, and CD 11b. In some aspects of the invention cord blood cells are used without purification by subset.

In another aspect of the invention, patients with valvular degeneration are treated with a therapeutically effective amount of placental stem cells, said stem cells may be identified based on expression of one or more antigens selected from a group comprising: Oct-4, Rex-1, CD9, CD13, CD29, CD44, CD166, CD90, CD105, SH-3, SH-4, TRA-1-60, TRA-1-81, SSEA-4 and Sox-2. In some aspects of the invention placental stem cells are used without purification by subset.

In another aspect of the invention, patients with valvular degeneration are treated with a therapeutically effective amount of bone marrow stem cells; said bone marrow stem cells comprised of bone marrow derived mononuclear cells. Said bone marrow stem cells may also be selected based upon ability to differentiate into one or more of the following cell types: endothelial cells, muscle cells, and neuronal cells. Said bone marrow stem cells may also be selected based on expression of one or more of the following antigens: CD34, c-kit, flk-1, Stro-1, CD105, CD73, CD31, CD146, vascular endothelial-cadherin, CD133 and CXCR-4. In one particular aspect, said bone marrow stem cells are selectively enriched for mononuclear cells expressing the protein marker CD133.

In another aspect of the invention, patients with valvular degeneration are treated with a therapeutically effective amount of amniotic fluid stem cells, wherein said amniotic fluid stem cells are isolated by introduction of a fluid extraction means into the amniotic cavity under ultrasound guidance. Said amniotic fluid stem cells may be selected based on expression of one or more of the following antigens: SSEA3, SSEA4, Tra-1-60, Tra-1-81, Tra-2-54, HLA class I, CD13, CD44, CD49b, CD105, Oct-4, Rex-1, DAZL and Runx-1 and lack of expression of one or more of the following antigens: CD34, CD45, and HLA Class II.

In another aspect of the invention, patients with valvular degeneration are treated with a therapeutically effective amount of neuronal stem cells that are selected based on expression of one or more of the following antigens: RC-2, 3CB2, BLB, Sox-2hh, GLAST, Pax 6, nestin, Muashi-1, NCAM, A2B5 and prominin.

In another aspect of the invention, patients with valvular degeneration are treated with a therapeutically effective amount of peripheral blood derived stem cells. Said peripheral blood derived stem cells may be characterized by expression of one or more markers selected from a group consisting of CD34, CXCR4, CD117, CD113, and c-met, and in some cases by ability to proliferate in vitro for a period of over 3 months. In some situations peripheral blood stem cells are purified based on lack of expression of differentiation associated markers, said markers selected from a group consisting of CD2, CD3, CD4, CD11, CD11a, Mac-1, CD14, CD16, CD19, CD24, CD33, CD36, CD38, CD45, CD56, CD64, CD68, CD86, CD66b, and HLA-DR.

In another aspect of the invention, patients with valvular degeneration are treated with a therapeutically effective amount of mesenchymal stem cells, said cells may be defined by expression of one or more of the following markers: STRO-1, CD105, CD54, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and in some situations lack of substantial levels of one or more of the following markers: HLA-DR, CD117, and CD45. In some aspects said mesenchymal stem cells are derived from a group selected of: bone marrow, adipose tissue, umbilical cord blood, placental tissue, peripheral blood mononuclear cells, differentiated embryonic stem cells, and differentiated progenitor cells.

In another aspect of the invention, patients with valvular degeneration are treated with a therapeutically effective amount of germinal stem cells, wherein said germinal stem cells may express markers selected from a group consisting of: Oct4, Nanog, Dppa5 Rbm, cyclin A2, Tex18, Stra8, Dazl, beta1- and alpha6-integrins, Vasa, Fragilis, Nobox, c-Kit, Sca-1 and Rex1.

In another aspect of the invention, patients with valvular degeneration are treated with a therapeutically effective amount of adipose tissue derived stem cells, wherein said adipose tissue derived stem cells may express markers selected from a group consisting of: CD13, CD29, CD44, CD63, CD73, CD90, CD166, Aldehyde dehydrogenase (ALDH), and ABCG2. In an alternative aspect adipose tissue derived stem cells derived as mononuclear cells extracted from adipose tissue that are capable of proliferating in culture for more than 1 month.

In another aspect of the invention, patients with valvular degeneration are treated with a therapeutically effective amount of exfoliated teeth derived stem cells, wherein said exfoliated teeth derived stem cells may express markers selected from a group consisting of: STRO-1, CD146 (MUC18), alkaline phosphatase, MEPE, and bFGF.

In another aspect of the invention, patients with valvular degeneration are treated with a therapeutically effective amount of hair follicle stem cells, wherein said hair follicle stem cells may express markers selected from a group consisting of: cytokeratin 15, Nanog, and Oct-4, in some aspects, said hair follicle stem cells are chosen based on capable of proliferating in culture for a period of at least one month. In other aspects, said hair follicle stem cell is selected based on ability to secrete one or more of the following proteins when grown in culture: basic fibroblast growth factor (bFGF), endothelin-1 (ET-1) and stem cell factor (SCF).

In another aspect of the invention, patients with valvular degeneration are treated with a therapeutically effective amount of dermal stem cells, wherein said dermal stem cells express markers selected from a group consisting of: CD44, CD13, CD29, CD90, and CD105. In some aspects, said dermal stem cells are chosen based on ability of proliferating in culture for a period of at least one month.

In another aspect of the invention, patients with valvular degeneration are treated with a therapeutically effective amount of parthenogenically derived stem cells, wherein said parthenogenically derived stem cells are generated by addition of a calcium flux inducing agent to activate an oocyte followed by enrichment of cells expressing markers selected from a group consisting of SSEA-4, TRA 1-60 and TRA 1-81.

In another aspect of the invention, patients with valvular degeneration are treated with a therapeutically effective amount of stem cells generated by reprogramming, said reprogramming being induced, for example, by nuclear transfer, cytoplasmic transfer, or cells treated with a DNA methyltransferase inhibitor, cells treated with a histone deacetylase inhibitor, cells treated with a GSK-3 inhibitor, cells induced to dedifferentiate by alteration of extracellular conditions, and cells treated with various combination of the mentioned treatment conditions. In certain aspects, nuclear transfer comprises introducing nuclear material to a cell substantially enucleated, the nuclear material deriving from a host whose genetic profile is sought to be dedifferentiated. In certain aspects, the cytoplasmic transfer comprises introducing cytoplasm of a cell with a dedifferentiated phenotype into a cell with a differentiated phenotype, such that the cell with a differentiated phenotype substantially reverts to a dedifferentiated phenotype. In certain aspects, the DNA demethylating agent is selected from a group consisting of: 5-azacytidine, psammaplin A, and zebularine. In certain aspects, the histone deacetylase inhibitor is selected from a group consisting of: valproic acid, trichostatin-A, trapoxin A and depsipeptide.

In another aspect of the invention, patients with valvular degeneration are treated with a therapeutically effective amount of side population cells. In certain aspects, the side population cells are identified based on expression multidrug resistance transport protein (ABCG2) or ability to efflux intracellular dyes such as rhodamine-123 and or Hoechst 33342. In certain aspects, the side population cells are derived from tissues such as pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In another aspect of the invention, patients with valvular degeneration are treated with a therapeutically effective amount of committed progenitor cells. In certain aspects, the committed progenitor cells can be selected from a group consisting of: endothelial progenitor cells, neuronal progenitor cells, and hematopoietic progenitor cells. In certain aspects, the committed endothelial progenitor cells are purified from the bone marrow or are purified from peripheral blood.

In certain aspects, the committed endothelial progenitor cells are purified from peripheral blood of a patient whose committed endothelial progenitor cells are mobilized by administration of a mobilizing agent or therapy. The mobilizing agent can be selected from a group consisting of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. The mobilization therapy can be selected from a group consisting of: exercise, hyperbaric oxygen, autohemotherapy by ex vivo ozonation of peripheral blood, and induction of SDF-1 secretion in an anatomical area outside of the bone marrow. In certain aspects, the committed endothelial progenitor cells express markers selected from a group consisting of: CD31, CD34, AC133, CD146 and flk1.

In certain aspects, the committed progenitor cells are committed hematopoietic cells and are purified from the bone marrow or from peripheral blood. In certain aspects the committed hematopoietic progenitor cells are purified from peripheral blood of a patient whose committed hematopoietic progenitor cells are mobilized by administration of a mobilizing agent or therapy. In certain aspects, the mobilizing agent is selected from a group consisting of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. In certain aspects, the mobilization therapy is selected from a group consisting of: exercise, hyperbaric oxygen, autohemotherapy by ex vivo ozonation of peripheral blood, and induction of SDF-1 secretion in an anatomical area outside of the bone marrow. In certain aspects, the committed hematopoietic progenitor cells express the marker CD133. In certain aspects, the committed hematopoietic progenitor cells express the marker CD34.

In another aspect of the invention, patients with valvular degeneration are treated with a therapeutically effective amount of side population cells, wherein said cells are identified based on expression multidrug resistance transport protein (ABCG2) or ability to efflux intracellular dyes such as rhodamine-123 and or Hoechst 33342. Said side population cells may be derived from tissues such as pancreatic tissue, liver tissue, muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue.

In another aspect of the invention, patients with valvular degeneration are treated with a therapeutically effective amount of committed progenitor cells, wherein said committed progenitor cells are selected from a group consisting of: endothelial progenitor cells, neuronal progenitor cells, and hematopoietic progenitor cells. In another aspect of the invention, committed progenitor cells are purified from peripheral blood of a patient whose committed endothelial progenitor cells are mobilized by administration of a mobilizing agent or therapy. Said mobilizing agent is selected from a group consisting of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. Said mobilization therapy is selected from a group consisting of: exercise, hyperbaric oxygen, autohemotherapy by ex vivo ozonation of peripheral blood, and induction of SDF-1 secretion in an anatomical area outside of the bone marrow.

In another aspect of the invention, valvular dysfunction is treated through enhancing the number of circulating stem cells in a patient in need thereof, said enhancement may be performed through administration of a mobilization agent, or mobilization therapy, said mobilizing agent may be selected from a group consisting of: G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA) reductase inhibitors and small molecule antagonists of SDF-1. Said mobilization therapy may be selected from a group consisting of: exercise, hyperbaric oxygen, autohemotherapy by ex vivo ozonation of peripheral blood, and induction of SDF-1 secretion in an anatomical area outside of the bone marrow.

In another aspect of the invention, valvular dysfunction is treated through concurrently using a cellular or stem cell therapy in combination with enhancing the anti-oxidant status of a patient in need thereof. Administration of antioxidant can be at a concentration sufficient to reduce oxidative stress from inhibiting the beneficial effects of the stem cells on valvular abnormality and/or dysfunction. The antioxidant may be administered prior to, concurrently or subsequent to administration of stem cells. Enhancement of antioxidant status may be performed through administration of an antioxidant, or combination of antioxidants, said antioxidant may be selected from a group consisting of: ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, and superoxide dismutase.

In another aspect of the invention, valvular dysfunction is treated through concurrently using a cellular or stem cell therapy in combination with enhancing the anti-oxidant status of a patient in need thereof in combination with administration of an antihypertensive agent. The antihypertensive agent may be administered prior to, concurrently or subsequent to administration of stem cells. The antihypertensive agent may be a vasodilator. Said antihypertensive agent can be selected from a group consisting of hydralazine, cadralazine, felodipine, sildenafil, and nifedipine. In some aspects an angiotensin converting enzyme (ACE) inhibitor administered, said ACE-inhibitor may be selected from a group consisting of captopril, benazepril, enalapril, lisinopril, fosinopril, ramipril, perindopril, quinapril, moexipril, and trandolapril. In some aspects of the invention an antibiotic agent, or agents capable of substantially inhibiting bacterial infections in the cardiac area may also be used, said antibiotic(s) may be selected from a group consisting of: amoxicillin, ampicillin, cefadroxil, cephalexin, azithromycin, clarithromycin, clindamycin, and cefazolin.

In some aspects of the invention, a chemoattractant agent or combination of agents are administered either proximally, or directly on the malfunctioning valve with the purpose of attracting therapeutic cell populations and activating said cell populations to regenerate valvular structures. The attracted therapeutic cell populations can be stem cells, committed progenitor cells, and differentiated cells. The attracted therapeutic cell populations can be endogenous, and can be endogenous and mobilized. In certain aspects, the cells can be exogenous, and the exogenous cells can be selected from a group consisting of: autologous, allogeneic, and xenogeneic cells. The chemoattractant may be administered in the form of a depot injected intravalvularly, or in proximity of said valve. Said depot capable of substantially localizing said chemoattractant is may be selected from a group consisting of: fibrin glue, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol. Furthermore, said chemoattracted useful for the practice of the current invention may be is selected from a group comprising: SDF-1, VEGF, RANTES, ENA-78, platelet derived factors, various isoforms thereof and small molecule agonists of VEGFR-1, VEGFR2, and CXCR4.

In another aspect of the invention, the chemoattractant is administered into the area in need, through transfection of a single or plurality of nucleotide(s) encoding said chemoattractant factor.

In another aspect of the invention valvular repair is performed by surgical means, followed by administration of cellular therapy, alone, or in combination with the therapeutic modalities claimed for the purposes of accelerating the healing process, inhibiting restenosis, and reducing need for rehospitalization and/or reoperation.

In another aspect of the invention cell administration is used, alone, or in combination with the therapeutics claimed, in order to allow accelerated endothelialization, and/or allow prolonged efficacious function of a prosthetic, or bioprosthetic graft.

In another aspect of the invention a prosthetic or bioprosthetic graft is embedded with a chemoattractant of therapeutic cells in order to allow accelerated endothelialization, and/or allow prolonged efficacious function of a prosthetic, or bioprosthetic graft.

In another aspect of the invention, therapeutic cells are administered via the coronary artery or arteries substantially proximal to area of the ventricle or atrium anticipated to undergo hypertrophy as a result of valvular dysfunction. Said administration may be performed by means known in the art, for example by balloon catheter, a Noga-Myostar device, and direct administration during surgery.

In another aspect of the invention, a chemoattractant is administered to the area anticipated to undergo hypertrophy. Said chemoattractant may in some aspects be administered by combination with a localizating agent. In some aspects mobilization of endogenous stem cells is used as part of this treatment combination. In other aspects, allogeneic or xenogeneic cells are administered.

In another aspect of the invention, therapeutic interventions as described in the claims of this invention are performed with the goals of: a) maintaining the cardiac-thoracic ratio lower than 0.64; b) maintaining fractional shortening>29%; c) maintaining end-systolic diameter<55 mg; d) maintaining the ratio of end-diastolic radius to myocardial wall thickness<4.0; d) maintaining left ventricular ejection fraction>0.45; and e) maintaining the cardiac index>2.2 L/min/m(2).

In another aspect of the invention, maternal stem cells are mobilized in a women impregnated with an offspring susceptible to a congenital valvular disease. Said mobilization is performed with the aim of enhance transfer of therapeutic maternal stem cells in the offspring and allowing endogenous repair of said congenital abnormality.

Also presented herein is a method of preventing eccentric myocardial hypertrophy comprising administration of cells, and/or products of said cells, and/or cell stimulatory compounds, and/or cardiac protective factors to an individual in need thereof.

In certain aspects, the eccentric myocardial hypertrophy is the result of valvular heart disease, a state in which suboptimal function of one or more of the following cardiac valves is present: aortic valve, pulmonary valve, tricuspid valve, and mitral valve. In certain aspects, the suboptimal function includes inability to properly close at the time when complete closing is physiological, said reason for improper closure is selected from a group consisting of: malcoaptation, perforation of said valve, and altered mechanical properties.

In certain aspects, stem cells, and/or committed progenitors, and/or differentiated cells are administered into the left ventricle at a concentration sufficient to prevent, and/or reduce left ventricular diastolic end volume. In certain aspects, the cells are administered locally into left ventricle myocardium. In certain aspects, the cells are administered via the coronary artery or arteries substantially proximal to area anticipated to undergo hypertrophy. In certain aspects, the cells are administered by a method selected from: a balloon catheter, a Noga-Myostar device, and direct administration during surgery. In certain aspects, a chemoattractant is administered to the area anticipated to undergo hypertrophy. The chemoattractant can be administered in combination with a localizing agent. In certain aspects, the administration of cells occurs through mobilization of endogenous cells.

In certain aspects of the above embodiments, therapeutic intervention is tailored to the individual left ventricular and valvular characteristics of the patient. Thus, in certain aspects, the therapeutic intervention is performed with the goals of: a) maintaining the cardiac-thoracic ration lower than 0.64; b) maintaining fractional shortening>29%; c) maintaining end-systolic diameter<55 mg; d) maintaining the ratio of end-diastolic radius to myocardial wall thickness<4.0; d) maintaining left ventricular ejection fraction>0.45; and e) maintaining the cardiac index>2.2 L/min/m(2)

Also presented herein is a method of treating congenital valvular dysfunction in an unborn child through stimulation of maternal stem cell mobilization as described herein.

Also presented herein is a method of treating stenotic valves using the stem cell therapy as described herein.

In certain aspects of the above embodiments, endogenous stem cells are activated for the treatment of valvular degeneration through administration of thalidomide or compounds related to thalidomide at a concentration and frequency therapeutic to valvular degeneration.

Also provided herein is a method of treating valvular degeneration through administration of a therapeutically sufficient concentration, at a therapeutically sufficient interval, of a supernatant derived from a stem cell population.

### DETAILED DESCRIPTION OF THE PREFERRED EMBODIMENT

The invention disclosed is centered on the ability of various cells to cause regeneration of cardiac structures, said regeneration being beneficial to patients suffering from valvular disease. Said cells may be stem cells, progenitor cells, committed progenitor cells, or cells with reparative properties. Accordingly, in the description of the invention, the words “stem cells”, “progenitor cells”, “committed progenitor cells”, or “cells with reparative properties” will be used interchangeably. Furthermore, in this application the term “valvular degeneration” will represent not only pathological or degenerative changes to the cardiac valves themselves, but will also represent pathology to cardiac structures that are associated with the valvular degenerative process. Within the definition of valvular degeneration falls, for example, annular degeneration, pathological myocardial alterations, segmental and global hypertrophy either of the concentric or eccentric form. Furthermore the term “valvular degeneration” will be interchangeably used with “valvular dysfunction” and will represent the same meaning.

The use of stem cells in treatment of valvular heart disease may take many embodiments and the description provided herein is only meant to be illustrative of some of the embodiments. One skilled in the art will realize that numerous modifications, concentrations, and additives may be used in the context of practicing the current invention without departing for the spirit of the teachings herein.

Stem cells may be used that are exogenous to the individual in need of therapy, or stem cells that are endogenously originating. In situations of exogenous stem cell administration, stem cells may be autologous, syngeneic, allogeneic, or xenogeneic. In some situations practicing the invention will require the use of mixtures between autologous, syngeneic, allogeneic, and xenogeneic. One example of such a situation is the “reprogramming” of autologous cells with cytoplasmic factors derived from syngeneic, allogeneic, or xenogeneic cells. Another example of such “chimeric cells” includes fusion of one portion of the autologous cell, with a portion of the syngeneic, allogeneic, or xenogeneic cell. These chimeric cells may be generated in situations where it is desired to endow certain properties to autologous cells, properties, such as enhanced proliferative or dedifferentiation states that are capable of induction by components of syngeneic, allogeneic, or xenogeneic cells. On the other hand, in some embodiments of the invention endogenous stem cells may be activated to proliferate by administration of various compounds to that patient, or by exposing said patient to specific therapeutic conditions such as administration of various radio frequencies. Other embodiments of the invention utilizing endogenous stem cells involve the mobilization of stem cells from one anatomical niche to systemic circulation, or into another specific anatomical niche. Mobilization may be caused by administration of factors capable of stimulating stem cell exodus from compartments such as the bone marrow, or by exposing said patient to therapeutic conditions such as exercise or hyperbaric oxygen.

The underlying theme of the invention teaches the use of stem cells for treatment of valvular degeneration. In addition to actually healing valves, or accelerating healing subsequent to an intervention, stem cells are also used within the context of the current invention for repairing and/or regenerating myocardial structures that are defective due to said valvular degeneration. One said structure is the aortic root. It is known that activation of various pathological processes, including enhanced fibrosis occurs during aortic root dilation (36, 37). In fact, circulating levels of matrix metalloproteases are associated with aortic root dilation (38). Accordingly, one aspect of the current invention is to promote physiological remodeling and decrease fibrosis through administration of therapeutically active stem cells. In this context, and also of actual valve repair, specific properties of stem cells that make them suitable for use in practicing the current invention are, inter alia: a) ability to decrease fibrosis (39, 40); b) induce regeneration of functional tissue (41); c) enhance function and contractility of existing tissues through secretion of soluble factors, as well as via membrane bound activities (42-44), and d) inhibit inflammation (45, 46). In the context of maintaining and re-forming appropriate ventricular/atrial dimensions during valvular disease, stem cells are useful therapeutically due to properties which include, inter alia: a) ability to increase contractility and compliance (47); b) ability to stimulate neoangiogenesis (48); c) ability to decrease fibrosis of the chamber (49, 50); d) ability to evoke myocardial regeneration (51); and e) inhibit inflammation (52-54). Although autologous and allogeneic stem cells have been used extensively in the area of myocardial infarction (55-59), dilated cardiomyopathy (60, 61), end-stage angina (55, 62), and to increase efficiency of coronary artery bypass grafting (63), both in preclinical and clinical situations, utilization of stem cells for treatment of valvular degeneration has not been performed. To date, the use of stem cells for valvular degeneration has been strictly limited to in vitro production of artificial valves (64).

When selecting stem cells for use in the practice of the current invention, several factors must be taken into consideration, such as: ability for ex vivo expansion without loss of therapeutic activity, ease of extraction, general potency of activity, and potential for adverse effects. Ex vivo expansion ability of stem cells can be measured using typical proliferation and colony assays known to one skilled in the art, while identification of therapeutic activity depends on functional assays that test biological activities. Biological activities of relevance in practicing the current invention are dependent on whether the patient is being treated for the purpose of regenerating afflicted valvular structure, whether the patient is being treated for the purpose of preventing/reversing chamber and other cardiac structures malformation, or whether stem cell therapy will be used for the purpose of accelerating healing post-intervention. For example, when the primary purpose of treatment is to increase valvular compliance and regeneration, it is important that stem cells possess properties that inhibit fibrosis and assist valvular interstitial cells to multiply. Stem cells may be tested for this function by culture of said stem cells in the presence of valvular interstitial cells. One method, used by Ku et al (65), of obtaining valvular interstitial cells from the aortic valve involves placing isolated valve leaflets (from autopsy samples) in a solution of Dulbecco's Modified Eagle Media (DMEM), containing 1000 U/ml of collagenase type II, agitating the mixture initially for approximately 5 minutes so that surface endothelial cells are detached (can assess by microscopy), washing said valve in phosphate buffered saline (PBS), mechanically dissociated said valve, and reincubating said valve with identical media for an additional 45 minutes at 37 Celsius. Subsequently fetal calf serum is added to the dissociated valve in order to block the collagenase II degradation, cells are washed in PBS, and cells are plated on 6-well tissue culture plates in the presence of DMEM media with 10% FCS, supplemented with 100 ug/ml penicillin and 100 U/ml streptomycin. Cells are incubated for approximately 4-7 days at 37 Celsius, with 5% carbon dioxide in a humidified incubator. Assessment of valvular interstitial cell phenotype is performed by examining expression of markers specific to said cells, such as vimentin. Confluent valvular cells may subsequently be cultured in the presence of varying amount of stem cells that are contemplated for human therapeutic use. The ability of added stem cells to increase fibrosis, alter viability, or morphology of said valvular interstitial cells during culture can be used as a means of assessing optimal stem cells for use in treatment of valvular degeneration. Ability of stem cells to alter the various mechanical properties of valvular interstitial cells may also be assessed. Methods for assaying response to mechanical stretch are described in Ku et al (65). If stem cell therapy is contemplated for the use of treating mitral stenosis, it is possible to use similar assays to assess ability of stem cells to inhibit osteoblastic transformation of valvular cells using systems described by Rajamannan et al (66).

In addition to functional in vitro assays, assessment of therapeutic activity can also be performed using surrogate assays, which detect markers associated with a specific therapeutic activity. Such markers include CD34 or CD133, which are associated with stem cell activity and ability to support angiogenesis (67). Other assays useful for identifying therapeutic activity of stem cell populations for use with the current invention include evaluation of production of factors associated with the therapeutic activity desired. For example, identification and quantification of production of FGF, VEGF, angiopoietin, or other such angiogenic molecules may be used to serve as a guide for approximating therapeutic activity in vivo if angiogenesis is the desired activity (68). Additionally, secretion of factors that inhibit valvular fibrosis or remodeling of either the valve or the myocardium may also be used as a marker for identification of cells that are useful for practicing the current invention.

In terms of assessing angiogenic potential means include, inter alia assessment of ability of stem cells for: a) ability to support endothelial cell proliferation in vitro using human umbilical vein endothelial cells or other endothelial populations. Assessment of proliferation may be performed using tritiated thymidine incorporation or by visually counted said proliferating endothelial cells. A viability dye such as MTT or other commercially available indicators may be used; b) Ability to support cord formation in subcutaneously implanted matrices. Said matrices, which may include Matrigel or fibrin gel, are loaded with cells generated as described above and implanted subcutaneously in an animal. Said animal may be an immunodeficient mouse such as a SCID or nude mouse in order to negate immunological differences. Subsequent to implantation formation of endothelial cords may be assessed visually by microscopy. In order to distinguish cells stimulating angiogenesis versus host cells responding to said cells stimulating angiogenesis, a species-specific marker, labeling of cells, or genetically labeled cells may be used; c) Ability to accelerate angiogenesis occurring in the embryonic chicken chorioallantoic membrane assay. Cells may be implanted directly, or via a matrix, into the chicken chorioallantoic membrane on embryonic day 9 and cultured for a period of approximately 2 days. Visualization of angiogenesis may be performed using in vivo microscopy; and d) Ability to stimulate neoangiogenesis in the hind limb ischemia model described above.

Assessment of the anti-inflammatory abilities of stem cells generated or isolated for potential clinical use may also be performed. Numerous methods are known in the art, for example they may include assessment of the putative anti-inflammatory cells to modulate immunological parameters in vitro. Putative anti-inflammatory cells may be co-cultured at various ratios with an immunological cell. Said immunological cell may be stimulated with an activatory stimulus. The ability of the putative anti-inflammatory cell to inhibit, in a dose-dependent manner, production of inflammatory cytokines or to augment production of anti-inflammatory cytokines, may be used as an output system of assessing anti-inflammatory activity. Additional output parameters may include: proliferation, cytotoxic activity, production of inflammatory mediators, or upregulation of surface markers associated with activation. Cytokines assessed may include: IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, TNF, IFN and RANKL. Inhibition of certain cytokines associated with the specific valvular abnormality may be more desired in certain situations. For example it is known that circulating levels of TNF-alpha are higher in patients with mitral stenosis (69), and it is postulated that TNF alpha is involved in subsequent cardiac remodeling through induction of matrix metalloproteases, (70), as well as onset of heart failure (71). Alternatively, upregulation of cytokines such as interleukin 10 has been associated with anti-inflammatory activities, and protection from heart failure (72). Accordingly in some embodiments of the invention it may be beneficial to select stem cells capable of inhibiting TNF alpha and upregulating interleukin 10. Specific immunological cells may be freshly isolated or may be immortalized cell lines. Said immunological cells may be: T cells, B cells, monocytes, macrophages, neutrophils, eosinophils, basophils, dendritic cells, natural killer cells, mast cells, natural killer T cells, and gamma delta-T cells. Said immunological stimuli may include an antibody, a ligand, a protein, or another cells. Examples including: crosslinking antibodies to T cell receptor, to costimulatory molecules such as CD28, to activation associated molecules such as CD69 or to receptors for stimulatory cytokines such as IL-2. Additional examples of inflammatory stimuli may include co-culture with allogeneic stimulator cells such as in mixed lymphocyte reactions, or may include stimulation with a non-specific activator such as a lectin. Specific lectins may include conconavalin-A, phytohemagluttinin, or wheat germ agglutinin. Other non-specific stimulators may be activators of the toll like receptor pathway such as lipopolysaccharide, CpG DNA motifs or bacterial membrane fractions.

In one embodiment of the invention, patients presenting with a valvular disease are treated at a timepoint where surgical, or percutaneous intervention is not immediately required. An example of such a situation is patients with aortic insufficiency (AI) who have chronic severe regurgitation, but have a left ventricular ejection fraction above 55% and the left ventricle end-diastolic dimension is below 55 mm (73). In the case of AI, patients are frequently placed on various medical treatments such as ACE inhibitors to decrease amount of remodeling. On reason for this is because volume overload in the case of AI is associated with secretion of angiotensis by stretched myocardial cells, which results in the in-series addition of sarcomeres and eccentric hypertrophy. Such patient populations where surgical or percutaneous intervention can be significantly delayed are one type of candidate for the invention disclosed herein. Other indications for therapy include mitral, pulmonic, or aortic stenosis, in which valvoplasty or other surgical/percutaneous intervention is not immediately required. Furthermore, conditions such as mitral valve, tricuspid valve, or pulmonic valve regurgitation in which immediate surgical/percutaneous intervention is not required are also suitable for practice of the current invention.

One method of practicing the invention involves systemic administration of umbilical cord blood stem cells with the goal of reducing remodeling, increasing annular integrity, and enhancing the level of leaflet coaptation or closing. Said cord blood stem cells may be administered as a heterogenous population of cells by the administration of cord blood mononuclear cells. Said cells may be isolated according to many methods known in the art. In one particular method, cord blood is collected from fresh placenta and mononuclear cells are purified by centrifugation using a density gradient such as Ficoll or Percoll. In another method cord blood mononuclear cells are isolated from contaminating erythrocytes and granulocytes by the Hetastarch with a 6% (wt/vol) hydroxyethyl starch gradient.

In one particular embodiment, cord blood is collected from placenta of full-term deliveries in a multiple bag system containing citrate phosphate dextrose buffer or another suitable anti-coagulant and processed within 24 hours of collection. Anticoagulated cord blood is diluted 1:1 with 2 mM EDTA/PBS. Mononuclear cells are separated by gradient centrifugation as known in the art. For example, centrifugation may be performed at 450 g for 30 min at room temperature using Ficoll-Hypaque density gradient solution. Cells are seeded at a density of 1×10⁶ cells/cm² into culture plates pre-coated with autologous serum. 12-20 hours after plating, the non-adherent cells are removed by washing the plate with phosphate buffered saline. Cells are passaged by trypsinization when confluence is reached and passaged as needed, approximately once every three days. Cells may be cultured in a wide variety of vessels such as bioreactors, flasks, plates, or other means. Culture medium useful for growing and expanding mesenchymal stem cells is widely known in the art and may include RMPI, DMEM, or MSCGM (Cambrex). When an appropriate number of cells is reached, cells are harvested and either cryopreserved for later use or administered into the patient in need of therapy. Methods of administration and subsequent patient assessment are well-known in the art and briefly reviewed in this disclosure. Cells are subsequently washed to remove contaminating debris, assessed for viability, and administered at a concentration and frequency sufficient to induce therapeutic benefit. Cord blood cells may be matched for the haplotype of the specific patient according to HLA status, or may be mismatched

The suitability of cord blood derived cells for use in the practice of the present invention is further supported by the fact that numerous patients to whom the invention is applicable to will not be able or willing to undergo the invasive process of bone marrow harvest. The use of allogeneic bone marrow is contemplated within the scope of the invention, however certain patients may not opt for this approach due to fear graft versus host disease development. The use of cord blood is therefore preferred for certain patients due to the known lower incidence of graft versus host disease when cord blood transplants are performed (74, 75). Additionally, the same two studies cited also report lower level of viral contamination and increased availability of donors (due to laxer HLA-matching criteria) in patients receiving cord blood as opposed to bone marrow grafts.

In another embodiment of the invention, cord blood stem cells are fractionated and the fraction with enhanced therapeutic activity is administered to the patient. Enrichment of cells with therapeutic activity may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the invention, it will be useful to select cord blood stem cells with a higher proliferative ability, whereas in other situations, a lower proliferative ability may be desired. In some embodiments of the invention, cells are directly injected into the area of need, such as in the mitral annulus for patients with mitral regurgitation, into the aortic annulus for patients with AI, or directly into degenerating valves. In other embodiments, cells are administered systemically and in this case with may be desirable for the administered cells to be relatively undifferentiated, so has to still possess homing activity to the area of need. In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for valvular degeneration, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

Certain desired activities can be endowed onto said cord blood stem cells prior to administration into the patient. In one specific embodiment cord blood cells may be “activated” ex vivo by a brief culture in hypoxic conditions in order to upregulate nuclear translocation of the HIF-1 transcription factor and endow said cord blood cells with enhanced angiogenic potential. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, such as homing to a chemotactic gradient, for example SDF-1, can be performed prior to administration of said cord blood cells into the patient. Assessment methods are known in the art and include measurement of angiogenic factors, ability to support viability and activity of cells associated with erectile function, as well as ability to induce regeneration of said cellular components associated with valvular function, myocardial remodeling, and/or inhibition of inflammation.

In addition to induction of hypoxia, other therapeutic properties can be endowed unto cord blood stem cells through treatment ex vivo with factors such as de-differentiating compounds, proliferation inducing compounds, or compounds known to endow and/or enhance cord blood cells to possess properties useful for the practice of the current invention. In one embodiment cord blood cells are cultured with an inhibitor of the enzyme GSK-3 in order to enhance expansion of cells with pluripotent characteristics while not increasing the rate of differentiation. In another embodiment, cord blood cells are cultured in the presence of a DNA methyltransferase inhibitor such as 5-azacytidine in order to endow a “de-differentiation” effect. In another embodiment cord blood cells are cultured in the presence of a differentiation agent that skews said cord blood stem cells to generate enhance numbers of cells which are useful for treatment of vascular degeneration after said cord blood cells are administered into a patient. For example, cord blood cells may be cultured in estrogen, hepatocyte growth factor, or various cardiotrophic factors for a brief period so that subsequent to administration, an increased number of cardio-reparative cells are generated.

Therapeutic dosage may be tailored to responsiveness of the patient, urgency of need for percutaneous/surgical intervention, as well as individual patient characteristics. Monitoring of patients with valvular degeneration is well-known in the art and includes inter alia standard diagnostic techniques such as echocardiogram, cardiac catheterization, assessment of pulmonary hypertension. More esoteric assessments are also included within the scope of the current invention. For example, in patients with mitral stenosis, where a high amount of inflammatory associated markers are present, such as c reactive protein, said marker can be used as a guide for determining the amount and frequency of therapy needed. Other measurements such as amount of cytokines in peripheral blood may also be used. Relevant cytokines include inter alia IL-1, TNF-alpha, and IL-10. In some embodiments of the invention, cord blood mononuclear cells that have not been purified for any particular cellular subset are injected intravenously at a concentration ranging from 1×10⁶ to 1×10⁹ per patient, more preferably at 1×10⁷ to 9×10⁸ per patient, and more preferably at approximately 5×10⁸ per patient.

In contrast to cord blood stem cells, placental stem cells may be purified directly from placental tissues, said tissues including the chorion, amnion, and villous stroma (76, 77). Allogeneic, or autologous stem cells may be used. In one embodiment of the invention, placental tissue is mechanically degraded in a sterile manner and treated with enzymes to allow dissociation of the cells from the extracellular matrix. Such enzymes include, but not restricted to trypsin, chymotrypsin, collagenases, elastase and/or hylauronidase. Suspension of placental cells are subsequently washed, assessed for viability, and may either be used directly for the practice of the invention by administration either locally or systemically. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations it will be desirable to expand cells before introduction into the human body. Expansion can be performed by culture ex vivo with specific growth factors (78, 79). The various embodiments of the invention described in this disclosure can also be applied for placental stem cells. Placental cells may be administered either locally or systemically into a patient with valvular degeneration.

Bone marrow stem cells may be used for the practice of the current invention either freshly isolated, purified, or subsequent to ex vivo culture. A typical step for collecting starting material for practicing one embodiment of the invention involves performing a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness and need for less anesthetic is the “mini-bone marrow harvest” (80). Said bone marrow harvest aspirate is used as a starting material for purification of cells with therapeutic activity in valvular degeneration. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of valvular degeneration. The various embodiments of the invention for other stem cells described in this disclosure can also be applied for bone marrow stem cells.

Amniotic fluid is routinely collected during amniocentesis procedures. One method of practicing the current invention is utilizing amniotic fluid derived stem cells for treatment of valvular degeneration. In one embodiment amniotic fluid mononuclear cells are utilized therapeutically in an unpurified manner. Said amniotic fluid stem cells are administered either locally or systemically in a patient suffering from valvular degeneration. In other embodiments amniotic fluid stem cells are substantially purified based on expression of markers such as SSEA-3, SSEA4, Tra-1-60, Tra-1-81 and Tra-2-54, and subsequently administered. In other embodiments cells are cultured, as described in U.S. Patent Application Publication 2005/0054093 (hereby incorporated by reference in its entirety), expanded, and subsequently infused into the patient. Amniotic stem cells are described in the following references (81-83). One particular aspect of amniotic stem cells that makes them amenable for use in practicing certain aspects of the current invention is their bi-phenotypic profile as being both mesenchymal and endothelial progenitors (82, 84). This property is useful for treatment of patients with valvular degeneration in which said mesenchymal progenitors allow for enhancement in valvular contractility, whereas said endothelial progenitors allow for neoangiogenesis and vascular repair. The use of amniotic fluid stem cells is particularly useful in situations such as ischemia associated mitral regurgitation and/or stenosis, in which hypoxia is known to perpetuate the process of tissue remodeling and eventual heart failure (85-88). The various embodiments of the invention for other stem cells described in this disclosure can also be applied for amniotic fluid stem cells.

A wide variety of stem cells are known to circulate in the periphery in cancer patients (89), patients with myocardial disease (90, 91), acute myocardial infarction patients (92), and healthy individuals (93). These include multipotent, pluripotent, and committed stem cells. In some embodiments of the invention mobilization of stem cells is induced in order to increase the number of circulating stem cells, so that harvesting efficiency is increased. Said mobilization allows for harvest of cells with desired properties for practice of the invention without the need to perform bone marrow puncture. A variety of methods to induce mobilization are known. Methods such as administration of cytotoxic chemotherapy, for example, cyclophosphamide or 5-fluoruracil are effective but not preferred in the context of the current invention due to relatively unacceptable adverse events profile. Suitable agents useful for mobilization include: granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), thrombopoietin (TPO), interleukin-11 (IL-11), insulin-like growth factor-1 (IGF-1), megakaryocyte growth and development factor (MGDF), nerve growth factor (NGF), hyperbaric oxygen, and 3-hydroxy-3-methyl glutaryl coenzyme A (HMG CoA) reductase inhibitors.

In a preferred embodiment, donors (either autologous or allegeneic) are mobilized by administration of G-CSF (filgrastim: neupogen) at a concentration of 10 ug/kg/day by subcutaneous injection for 2-7 days, more preferably 4-5 days. Peripheral blood mononuclear cells are collected using an apheresis device such as the AS104 cell separator (Fresenius Medical). 1-40×10⁹ mononuclear cells are collected, concentrated and injected into the area of valvular degeneration. Alternatively, cells may be injected systemically, or in an area proximal to the region pathology associated with valvular degeneration. In situations where ischemia is identified as causative, or a consequence of valvular degeneration, cellular administration may be performed within the context of the current invention. Methods of identification of such areas of ischemia is routinely known in the art and includes the use of techniques such as nuclear or MRI imagining. Variations of this procedure may include steps such as subsequent culture of cells to enrich for various populations known to possess angiogenic and/or anti-inflammatory, and/or anti-remodeling, and/or regenerative properties. Additionally cells may be purified for specific subtypes before and/or after culture. Treatments can be made to the cells during culture or at specific timepoints during ex vivo culture but before infusion in order to generate and/or expand specific subtypes and/or functional properties. The various embodiments of the invention for other stem cells described in this disclosure can also be applied for circulating peripheral blood stem cells.

In one embodiment, mesenchymal stem cells are generated through culture or utilized as freshly isolated cells. For example, U.S. Pat. No. 5,486,359 (hereby incorporated by reference in its entirety) describes methods for culturing and expanding mesenchymal stem cells, as well as providing antibodies for use in detection and isolation. U.S. Pat. No. 5,942,225 (hereby incorporated by reference in its entirety) teaches culture techniques and additives for differentiation of such stem cells which can be used in the context of the present invention to produce increased numbers of cells with angiogenic capability. Although U.S. Pat. No. 6,387,369 (hereby incorporated by reference in its entirety) teaches use of mesenchymal stem cells for regeneration of cardiac tissue, said application is limited in scope to cardiac chamber muscle and no claims are directed towards valvular tissue or valvular regeneration. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1 (60). Said factors are beneficial in the therapy of valvular degeneration within the context of the current invention.

Mesenchymal stem cells are classically obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue (94), placenta (77, 95), scalp tissue (96) and cord blood (97). A recent study compared mesenchymal stem cells from bone marrow, cord blood and adipose tissue in terms of colony formation activity, expansion potential and immunophenotype. It was demonstrated that all three sources produced mesenchymal stem cells with similar morphology and phenotype. Ability to induce colony formation was highest using stem cells from adipose tissue and interestingly in contrast to bone marrow and adipose derived mesenchymal cells, only the cord blood derived cells lacked ability to undergo adipocyte differentiation. Proliferative potential was the highest with cord blood mesenchymal stem cells which were capable of expansion to approximately 20 times, whereas cord blood cells expanded an average of 8 times and bone marrow derived cells expanded 5 times (98). Accordingly, one skilled in the art will understand that mesenchymal stem cells for use with the present invention may be selected upon individual patient characteristics and the end result sought. For example, if autologous mesenchymal stem cells are available in the form of adipocyte-derived cells, it will be more useful and beneficial to utilize this source instead of allogeneic cord-blood derived cells. Alternatively, cord blood derived mesenchymal stem cells may be more advantageous for use in situations where autologous cells are not available, and expansion is sought. The ability of mesenchymal stem cells from the cord blood to expand in vitro also allows the possibility of genetically modifying these cells in order to: a) decrease immunogeneicity; b) enhance angiogenic potential; and c) augment survival following administration. However it should be noted that such ex vivo manipulation is applicable to all cell types described in the current application.

In situations where a decrease in immunogenicity is sought, cells may be transfected using immune suppressive agents. Said agents include soluble factors, membrane-bound factors, and enzymes capable of causing localized immune suppression. Examples of soluble immune suppressive factors include: IL-4 (99), IL-10 (100), IL-13 (101), TGF-b (102), soluble TNF-receptor (103), and IL-1 receptor agonist (104). Membrane-bound immunoinhibitor molecules that may be transfected into stem cells for use in practicing the current invention include: HLA-G (105), FasL (106), PD-IL (107), Decay Accelerating Factor (108), and membrane-associated TGF-b (109). Enzymes which may be transfected in order to cause localized immune suppression include indolamine 2,3 dioxygenase (110) and arginase type II (111). In order to optimize desired immune suppressive ability, a wide variety of assays are known in the art, including mixed lymphocyte culture, ability to generate T regulatory cells in vitro, and ability to inhibit natural killer or CD8 cell cytotoxicity.

In situations where increased angiogenic potential of said mesenchymal stem cells is desired, mesenchymal stem cells may be transfected with genes such as VEGF (112), FGF1 (113), FGF2 (114), FGF4 (115), FrzA (116), and angiopoietin (117). Ability to induce angiogenesis may be assessed in vitro prior to administration of said transfected cells in vivo. Methods of assessing in vitro angiogenesis stimulating ability are well known in the art and include measuring proliferation of human umbilical vein derived endothelial cells.

Since one of the problems of cell therapy in general is viability of the infused cells subsequent to administration, it may be desired in some forms of the invention to transfect mesenchymal cells with genes protecting said cells from apoptosis. Anti-apoptotic genes suitable for transfection may include bcl-2 (118), bcl-xl (119), and members of the XIAP family (120). Alternatively it may be desired to increase the proliferative lifespan of said mesenchymal stem cells through transfection with enzymes associated with anti-senescence activity. Said enzymes may include telomerase or histone deacetylases. The various embodiments of the invention for other stem cells described in this disclosure can also be applied for mesenchymal stem cells.

Adipose derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 (hereby incorporated by reference in its entirety) describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently filtered using a cell strainer, for example a 100 μm nylon cell strainer in order to remove debris. Filtered cells are subsequently centrifuged again at approximately 700-2000 g for 5-15 minutes and resuspended at a concentration of approximately 1×10⁶/cm² into culture flasks or similar vessels. After 10-20 hours of culture non-adherent cells are removed by washing with PBS and remaining cells are cultured at similar conditions as described above for culture of cord blood derived mesenchymal stem cells. Upon reaching a concentration desired for clinical use, cells are harvested, assessed for purity and administered in a patient in need thereof as described above. The various embodiments of the invention for other stem cells described in this disclosure can also be applied for adipose derived stem cells.

Deciduous teeth (baby teeth) have been recently identified as a source of pluripotent stem cells with ability to differentiate into endothelial, neural, and bone structures. Said pluripotent stem cells have been termed “stem cells from human exfoliated deciduous teeth” (SHED). One of the embodiments of the current invention involves utilization of this novel source of stem cells for the treatment of valvular degeneration. In one embodiment of the invention, SHED cells are administered systemically or locally into a patient with valvular degeneration at a concentration and frequency sufficient for induction of therapeutic effect. SHED cells can be purified and used directly, certain sub-populations may be concentrated, or cells may be expanded ex vivo under distinct culture conditions in order to generate phenotypes desired for maximum therapeutic effect. Growth and expansion of SHED has been previously described by others. In one particular method, exfoliated human deciduous teeth are collected from 7- to 8-year-old children, with the pulp extracted and digested with a digestive enzyme such as collagenase type I. Concentrations necessary for digestion are known and may be, for example 1-5 mg/ml, or preferable around 3 mg/ml. Additionally dispase may also be used alone or in combination, concentrations of dispase may be 1-10 mg/ml, preferably around 4 mg/ml. Said digestion is allowed to occur for approximately 1 h at 37° C. Cells are subsequently washed and may be used directly, purified, or expanded in tissue culture. Recently, the commercial business of tooth stem cell banking has been announced at the website bioeden dot com. The various embodiments of the invention for other stem cells described in this disclosure can also be applied for exfoliated teeth stem cells.

The bulge area of the hair follicle bulge is an easily accessible source of pluripotent mesenchymal-like stem cells. One embodiment of the current invention is the use of hair follicle stem cells for treatment of valvular degeneration. Said cells may be used therapeutically once freshly isolated, or may be purified for particular sub-populations, or may be expanded ex vivo prior to use. Purification of hair follicle stem cells may be performed from cadavers, from healthy volunteers, or from patients undergoing plastic surgery. Upon extraction, scalp specimens are rinsed in a wash solution such as phosphate buffered saline or Hanks and cut into sections 0.2-0.8 cm. Subcutaneous tissue is de-aggregated into a single cell suspension by use of enzymes such as dispase and/or collagenase. In one variant, scalp samples are incubated with 0.5% dispase for a period of 15 hours. Subsequently, the dermal sheath is further enzymatically de-aggregated with enzymes such as collagenase D. Digestion of the stalk of the dermal papilla, the source of stem cells is confirmed by visual microscopy. Single cell suspensions are then treated with media containing fetal calf serum, and concentrated by pelletting using centrifugation. Cells may be further purified for expression of markers such as CD34, which are associated with enhanced proliferative ability. In one embodiment of the invention, collected hair follicle stem cells are induced to differentiate in vitro into neural-like cells through culture in media containing factors such as FGF-1, FGF-2, NGF, neurotrophin-2, and/or BDNF. Confirmation of neural differentiation may be performed by assessment of markers such as Muhashi, polysialyated N-CAM, N-CAM, A2B5, nestin, vimentin glutamate, synaptophysin, glutamic acid decarboxylase, serotonin, tyrosine hydroxylase, and GABA. Said neuronal cells may be administered systemically, or locally in a patient with valvular degeneration. Differentiation towards other phenotypes may also be performed within the context of the current invention. The various embodiments of the invention for other stem cells described in this disclosure can also be applied for hair follicle stem cells.

Parthenogenically derived stem cells can be generated by addition of a calcium flux inducing agent to activate oocytes, followed by purifying and expanding cells expressing embryonic stem cell markers such as SSEA-4, TRA 1-60 and/or TRA 1-81. Said parthenogenically derived stem cells are totipotent and can be used in a manner similar to that described for embryonic stem cells in the practice of the current invention. One specific methodology for generation of parthenogenically derived stem cells involves maturing oocytes by culture 36 hour in CMRL-1066 media supplemented with 20% FCS, 10 units/ml pregnant mare serum, 10 units/ml HCG, 0.05 mg/ml penicillin, and 0.075 mg/ml streptomycin. Mature metaphase II eggs are subsequently activated with calcium flux by incubation with 10 uM ionomycin for 8 minutes, followed by culture with 2 mM 6-dimethylaminopurine for 4 hours. The inner cell mass is subsequently isolated by immunosurgical technique and cells are cultured on a feeder layer in a manner similar to culture of embryonic stem cells (121). The various embodiments of the invention for other stem cells described in this disclosure can also be applied for parthenogenically derived stem cells.

Reprogramming of non-stem cells to endow them with stem cell characteristics can generate stem cells for use in the practice of the current invention. The advantage of reprogramming cells is that ability to withdraw autologous cells, which may have limited stem cell potential, endow said autologous cells with stem cell, or stem cell-like, properties, and reintroduce said autologous cells into the patient. In one embodiment, generation of a highly potent, undifferentiated source of stem cells is required for treatment of a patient with advanced valvular disease. Upon examination of various therapeutic properties of conventionally accessible stem cells, as disclosed in the invention, it is decided that said conventionally accessible cells are not of sufficient potency. Accordingly, the invention discloses methods of reprogramming autologous bone marrow cells using cytoplasmic extracts of embryonic stem cells in order to “rejuvenate” said bone marrow cells so as to increase pluripotency and self-renewal activity. In one embodiment, CD34+ bone marrow cells are purified from the patient in need of therapy. Said cells are prepared for reprogramming using embryonic stem cell cytoplasmic extracts. Said extracts are prepared according a modification to the method of Collas in U.S. Patent Application Publication 2002/0142397 (hereby incorporated by reference in its entirety). Briefly, interphase cultured embryonic stem cells of the H-1 line are harvested by trypsinization and washed by centrifugation at 500 g for 10 minutes in a 10 ml conical tube at 4 Celsius. The supernatant is discarded, and the cell pellet is resuspended in a total volume of 50 ml of cold PBS. The cells are centrifuged at 500 g for 10 minutes at 4 Celsius. This washing step is repeated, and the cell pellet is resuspended in approximately 20 volumes of ice-cold interphase cell lysis buffer (20 mM Hepes, pH 8.2, 5 mM MgCl.sub.2, 1 mM DTT, 10 .mu.M aprotinin, 10 .mu.M leupeptin, 10 .mu.M pepstatin A, 10 .mu.M soybean trypsin inhibitor, 100 .mu.M PMSF, and optionally 20 .mu.g/ml cytochalasin B). The cells are sedimented by centrifugation at 800 g for 10 minutes at 4 Celsius. The supernatant is discarded, and the cell pellet is carefully resuspended in no more than one volume of interphase cell lysis buffer. The cells are incubated on ice for one hour to allow swelling of the cells. The cells are lysed by sonication using a tip sonicator. Cell lysis is performed until at least 90% of the cells and nuclei are lysed, which is assessed using phase contrast microscopy. The sonication time required to lyse at least 90% of the cells and nuclei may vary depending on the type of cell used to prepare the extract. Accordingly, microscopic evaluation of cellular morphology is performed to assess degree of sonication needed. The cell lysate is placed in a 1.5-ml centrifuge tube and centrifuged at 10,000 to 15,000 g for 15 minutes at 4 Celsius using a table top centrifuge. The tubes are removed from the centrifuge and immediately placed on ice. The supernatant is carefully collected using a 200 .ul pipette tip, and the supernatant from several tubes is pooled and placed on ice. This cell extract is then aliquoted into 20 .ul volumes of extract per tube on ice. The tube is then overlayed with mineral oil to the top. The extract is centrifuged at 200,000 g for three hours at 4 Celsius to sediment membrane vesicles contained. At the end of centrifugation, the oil is discarded. The supernatant is carefully collected, pooled if necessary, and placed in a cold 1.5 ml tube on ice. This supernatant quantified for protein content and is referred to as the “cellular extract” that will be used for the reprogramming of cells. CD34+ cells are then permeabilized temporarily by incubation in Streptolysin O solution (see, for example, Maghazachi et al., 1997 and references therein) for 15-30 minutes at room temperature. After incubation, said cells are washed by centrifugation at 400.g for 10 minutes. This washing step is repeated twice by resuspension and sedimentation in PBS. The permeabilized CD34 stem cells are suspended in the embryonic stem cell derived reprogramming cytoplasmic extract (generated as described above) at a concentration of 300 cells per uL. An ATP generating system (2 mM ATP, 20 mM creatine phosphate, 50 .mu.g/ml creatine kinase) and 100 .mu.M GTP are added to the extract, and the reaction is incubated at 30-37 Celsius for up to two hours to promote translocation of factors from the extract into the cell and active nuclear uptake or chromosome-binding of factors. The reprogrammed cells are centrifuged at 800 g, washed by resuspension, and centrifugation at 400 g in PBS. The cells are resuspended in culture medium containing 20-30% fetal calf serum (FCS) and incubated for 1-3 hours at 37.degree. C. in a regular cell culture incubator to allow resealing of the cell membrane. The cells are then washed in regular warm culture medium (10% FCS) and cultured further in DMEM media, supplemented with IL-3 (20 ng/ml), IL-6 (250 ng/ml), SCF (10 ng/ml), TPO (250 ng/ml), and flt3-L (100 ng/ml). Media is exchanged 2-3 times per week. After 14 days of culture, cells are assessed by flow cytometry for markers of increased pluripotency and assessed for therapeutic activity suggestive of their in vivo ability to induce repair/regeneration of valvular degeneration. Said cells may be further cultured with various agents, or under various conditions in order to increase properties known to be therapeutic in the treatment of valvular degeneration. Alternatively, cells may be administered directly into said patient either locally or systemically. Other means of reprogramming stem cells are also described by Abuljadayel in U.S. Patent Application Publications 2003/0166272, 2003/0138947, 2002/0076812, 2001/0024826, and U.S. Pat. No. 6,090,625 (each of which is hereby incorporated by reference in its entirety). The various embodiments of the invention for other stem cells described in this disclosure can also be applied for reprogrammed stem cells.

For use in the context of the present invention, embryonic stem cells possess certain desirable properties, such as the ability to differentiate to almost every cell comprising the host. Additionally, embryonic stem cells secrete numerous factors capable of inhibiting the processes of valvular degeneration. Unfortunately, certain drawbacks exist that limit the utility of this cell type for widespread therapeutic implementation. The potential for carcinogenicity is apparent in that human embryonic stem cells administered to immunocompromised mice leads to formation of teratomas (122). Accordingly, for use in the current invention, embryonic stem cells have to be either differentiated into a stem cell, or a progenitor cell that is not capable of forming tumors. Cells useful the practice of the current invention should not differentiate in a substantial amount in an uncontrolled manner or into tissue which is pathological to the patient's well being. Although several technologies are currently being tested for selecting embryonic stem cells that do not cause teratomas, these methods are still in their infancy (123). Therefore, one method of utilizing embryonic stem cells for the practice of this invention is to encapsulate said embryonic stem cells, or place said cells into a permeable barrier so as to allow for secretion of therapeutic factors elaborated by said cells without the risk of causing cancer or undesired tissue growth. The use of said encapsulation technology has been successful in “semi-isolating” cells with therapeutic potential from the body, for examples of this the practitioner of the invention is referred to work on microencapsulation of islets for treatment of diabetes, in which cases xenogeneic islets are used (124, 125), or other systems of therapeutic cellular xenograft therapy (126-128). Said encapsulated cells may be administered systemically, or in a preferred embodiment locally in an area proximal to the main pathology of valvular degeneration, such as the various cardiac and epicardiac structures. Alternatively, encapsulated cells may be placed in a removable chamber in subcutaneous tissue similarly to the one described in U.S. Pat. No. 5,958,404, hereby incorporated by reference in its entirety. The advantages of using a removable chamber is that administration of cell therapy is not a permanent intervention and may be withdrawn upon achievement of desired therapeutic effect, or at onset of adverse effects.

For the practice of the invention, the practitioner is referred to the numerous methods of generating embryonic stem cells that are known in the art. Patents describing the generation of embryonic stem cells include U.S. Pat. No. 6,506,574 to Rambhatla, U.S. Pat. No. 6,200,806 to Thomson, U.S. Pat. No. 6,432,711 to Dinsmore, and U.S. Pat. No. 5,670,372 to Hogan, each of which is hereby incorporated by reference in its entirety. In one embodiment of the invention, embryonic stem cells are differentiated into endothelial progenitor cells in vitro, followed by administration to a patient in need of therapy at a concentration and frequency sufficient to ameliorate or cure valvular degeneration. Differentiation into endothelial progenitors may be performed by several means known in the art (129). One such means includes generation of embryoid bodies through growing human embryonic stem cells in a suspension culture. Said embryoid bodies are subsequently dissociated and cells expressing endothelial progenitor markers are purified (130). Purification of endothelial cells from embryoid bodies can be performed using, of example, selection for PECAM-1 expressing cells. Purified cells can be expanded in culture and used for injection. Another alternative method of generating endothelial progenitors from embryonic stem cells involves removing media from embryonic stem cells a period of time after said embryonic stem cells are plated and replacing said media with a media containing endothelial-differentiating factors. For example, after plating of embryonic stem cells for a period between 6 and 48 hours, but more preferably between 20 and 24 hours, the original media in which embryonic stem cells were cultured is washed off the cells and endothelial cell basal medium-2 (EBM2), with 5% fetal calf serum, VEGF, bFGF, IGF-1, EGF, and ascorbic acid is added to the cells. This combination is commercially available (EGM2-MV Bullet Kit; Clonetics/BioWhittaker, Walkersville, Md.). By culturing the embryonic stem cells for 20-30 days in the EGM2 medium, with changing of media every 3 to 5 days, a population of endothelial progenitors can be obtained. For such cells to be useful in the practice of the present invention, functionality of said endothelial precursors, and their differentiated progeny must be assessed. Methods of assessing endothelial function include testing their ability to produce and respond to NO, as well as ability to form cord-like structures in Matrigel, and/or form blood vessels when injected into immunocompromised mice (131). Injection of cells can be performed systemically, with the goal of injected cells homing to cardiac tissues associated with valvular degeneration, or alternatively administration may be local, via surgical or percutaneous administration. In variations of the invention where endothelial progenitors/endothelial cells are administered systemically, the local administration of an endothelial progenitor/endothelial cell chemoattractant factor may be used in order to increase the number of cells homing to the area of need. Said chemoattractant factors may include SDF-1 and/or VEGF, various isoforms thereof and small molecule agonists of the VEGFR-1 and/or VEGFR2, and/or CXCR4. Localization of said chemotactic factors to the area associated with valvular degeneration, or the valve itself may be performed using agents such as fibrin glue or certain delivery polymers known to one who is skilled in the art, these may include: polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol. Acceptable carriers, excipients, or stabilizers are also contemplated within the current invention, said carriers, excipients and stabilizers being relatively nontoxic to recipients at the dosages and concentrations employed, and may include buffers such as phosphate, citrate, and other organic acids; antioxidants including ascorbic acid, n-acetylcysteine, alpha tocopherol, and methionine; preservatives such as hexamethonium chloride; octadecyldimethylbenzyl ammonium chloride; benzalkonium chloride; phenol, benzyl alcohol, or butyl; alkyl parabens such as methyl or propyl paraben; catechol; resorcinol; cyclohexinol; 3-pentanol; and me-cresol); low molecular weight polypeptides; proteins, such as gelatin, or non-specific immunoglobulins; amino acids such as glycine, glutamine, asparagine, histidine, arginine, or lysine; monosaccharides, disaccharides, and other carbohydrates including glucose, mannose, or dextrins; chelating agents such as. EDTA; sugars such as sucrose, mannitol, trehalose, or sorbitol; salt-forming counter-ions such as sodium; metal complexes. Chemoattraction of cells with stem cell-like properties has been described in U.S. Patent Application Publication 2006/0003312 to Blau, hereby incorporated by reference in its entirety.

In another embodiment embryonic stem cells are differentiated into a desired phenotype microencapsulated so as to retain viability and ability to produce growth factors, while at the same time escaping immune mediated killing. This may be accomplished using known microencapsulation methods described in the art, such as described in U.S. Pat. No. 7,041,634 to Weber et al, or U.S. Patent Application Publication 2004/0136971 to Scharp et al, each of which is hereby incorporated by reference in its entirety. Additionally, embryonic stem cells may be irradiated either prior to, or subsequent to, encapsulation so as to block ability to proliferate while retaining growth factor producing activity.

In another embodiment embryonic stem cells are grown on the outside of a hollow-fiber filter which is connected to a continuous extracorporeal system. Said hollow-fiber system contains pores in the hollow fiber of sufficient size so has to allow exchange of proteins between circulating blood cells and cultured cells on the outside of the hollow fibers, without interchange of host cells with the embryonic stem cells. The various embodiments of the invention for other stem cells described in this disclosure can also be applied for embryonic stem cells.

Cells expressing the ability to efflux certain dyes, including but not limited to rhodamin-123 are associated with stem cell-like properties (132). Said cells can be purified from tissue subsequent to cell dissociation, based on efflux properties. Accordingly, in one embodiment of the current invention, tissue derived side population cells may be utilized either freshly isolated, sorted into subpopulations, or subsequent to ex vivo culture, for the treatment of valvular degeneration. For use in the invention, side population cells may be derived from tissues such as pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, and mesentery tissue. Purification of side population cells can be performed, in one embodiment, by resuspending dissociated cardiac valve cells at 10⁶ cells/ml, and staining with 6.0 μg/ml of Hoechst 33342 in calcium- and magnesium-free HBSS+ (supplemented with 2% FCS, 10 mM Hepes, and 1% penicillin/streptomycin) medium for 90 min at 37° C. Cells are then run on a flow cytometer and assessed for efflux of Hoechst 33342. Purified cells may be assessed for ability to form cardiac spheres, this may be performed by suspending said side population cells at a density of 1-2×106 cells/ml in 10-cm uncoated dishes in DME/M 199 (1:1) serum-free growth medium containing insulin (25 μg/ml), transferin (100 μg/ml), progesterone (20 nM), sodium selenate (30 nM), putrescine (60 nM), recombinant murine EGF (20 ng/ml), and recombinant human FGF2. Half of the medium is changed every 3 d. Passaging may be performed using 0.05% trypsin and 0.53 mM EDTA-4Na every 7-14 d. Cardiospheres are then dissociated into a single-cell suspension then used either for therapeutic purposes, or for evaluating ability to inhibit valvular degeneration in vitro or in animal models. These methods have been described in other publications to which the practitioner of the invention is referred to (133-135). The various embodiments of the invention for other stem cells described in this disclosure can also be applied for side population stem cells.

It is known that supernatants of stem cells have a variety of therapeutic properties. Although U.S. Patent Application Publication 2006/0057722 (hereby incorporated by reference in its entirety) discloses therapeutic angiogenesis properties of bone marrow supernatant with a filing date of Sep. 7, 2005, a publication from 2001 discloses angiogenic properties of bone marrow cells (136). Additionally activation of endogenous stem cells (137), anti-inflammatory properties have also been described in supernatants of stem cells (U.S. Pat. No. 6,231,893, hereby incorporated by reference in its entirety). Accordingly, supernatants of the various stem cells described in this disclosure may be utilized as a therapeutic in themselves, or as an adjuvant to administration of stem cells.

A specific embodiment of the current invention is the use of stem cell supernatant as a therapy for valvular degeneration. Specific embodiments include identification of substantially purified fractions of said supernatant capable of inducing various therapeutic activities known to be beneficial in patients with valvular degeneration. Identification of such therapeutically active fractions may be performed using methods commonly known to one skilled in the art, and includes separation by molecular weight, charge, affinity towards substrates and other physico-chemical properties. In one particular embodiment, supernatant of stem cell cultures is harvested substantially free from cellular contamination by use of centrifugation or filtration. Supernatant may be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of stem cell supernatant may be passed through each cartridge before elution. After washing the cartridges material adsorbed is eluted with 3 ml methanol, evaporated under a stream of nitrogen, redissolved in a small volume of methanol, and stored at 4.degree. C. Before testing the eluate for activity in vitro, the methanol is evaporated under nitrogen and replaced by culture medium. Said C18 cartridges are used to adsorb small hydrophobic molecules from the stem cell culture supernatant, and allows for the elimination of salts and other polar contaminants. It may, however be desired to use other adsorption means in order to purify certain compounds from the stem cell supernatant. Said concentrated supernatant may be assessed directly for biological activities useful for the practice of this invention, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically. In some embodiments, the various fractions are tested using in vitro models of fibrosis, inflammation, and/or remodeling prior to administration in humans in order to optimize therapeutic activity. In other aspects, therapeutic intervention using said fractions may be performed in animal models of valvular degeneration.

Another embodiment of the invention is a pharmaceutical preparation comprising a stem cell derived supernatant, either fractionated or unfractionated generated in a Good Manufacturing Practices/Good Tissue Practices environment that will allow it to be suitable for clinical use. In order to allow for regulatory approval, said supernatant is generated in an environment that is sterile, using a tissue culture media that does not contain animal proteins or undefined components. Such a media may be X-VIVO 10 or other clinically applicable medias. Other suitable liquid may be used, as long as conditions of sterility and GMP are practiced, such as for example a buffer, water such as isotonic water, growth medium, for example Iscove's modified Dulbecco's Media (IMDM) media, DMEM, KO-DMEM, DMEM/F12, RPMI 1640 medium, McCoy's 5A medium, minimum essential medium alpha medium (alpha.-MEM), F-12K nutrient mixture medium (Kaighn's modification, F-12K), X-vivo 20, Stemline, CC100, H2000, Stemspan, MCDB 131 Medium, Basal Media Eagle (BME), Glasgow Minimum Essential Media, Modified Eagle Medium (MEM), Opti-MEM I Reduced Serum Media, Waymouth's MB 752/1 Media, Williams Media E, Medium NCTC-109, neuroplasma medium, BGJb Medium, Brinster's BMOC-3 Medium, CMRL Medium, CO2-Independent Medium, and Leibovitz's L-15 Media or other liquid as determined by one of skill in the art. Stem cells are incubated under culture conditions for a period of time sufficient to allow production of therapeutic factors useful for treatment of patients with valvular degeneration. Cells may be incubated at various concentration and cells may be cultured under certain conditions in order to increase production of therapeutic factors. Said conditions include inter alia alteration in temperature, alternation in oxygen/carbon dioxide content, alternations in turbidity of said media, or exposure to small molecules modifiers of cell cultures such as nutrients, inhibitors of certain enzymes, stimulators of certain enzymes, inhibitors of histone deacytase activity such as valproic acid (138), trichostatin-A (139), trapoxin A (140), or Depsipeptide (141, 142), inhibitors of DNA methyltranserase activity such as 5-azacytidine, or inhibitors of the enzyme GSK-3 (143). In some embodiments stem cells will be transfected with genes to allow enhanced expression of factors therapeutic for valvular degeneration. Transfection may be performed with, for example, the hypoxia inducible factor-1, which is known to enhance secretion of angiogenically stimulatory factors such as VEGF (144). Supernatant from stem cells is either fractionated based on biological activities, as described above, or is directly concentrated. In one embodiment, concentration is performed by first passing culture supernatant over a sterile filter, such as a filter with a cut-off of 0.2 microns, and secondly utilizing a molecular weight filter such as an Amicon 3000 Stir Cell to reduce the volume and simultaneously remove low molecular weight salts. Other means to concentrate biologically active substances are widely known in the art and include membrane dialysis, lyophilization prior to membrane dialysis, and column chromatography. Subsequent to concentration and quantification of biological activity is performed using assays that determine amount of activity that would be therapeutic to valvular degeneration. For practical use, will be advantageous to formulate compositions in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms of the invention are dependent upon the amount of a compound necessary to stimulate the therapeutic activity, which is being sought. Actual formulation of the supernatant is performed in agreement with standard practices that are known to one skilled in the art. These are well known in the art and the one chosen is based upon the route of administration that will be used, as well as specific pharmacokinetic properties that are desired. For example, the preferred embodiment the supernatant is an injectable, more preferred an injection into the specific area requiring regeneration of stem cells, or other cells necessary for therapeutic effect in valvular degeneration. Several embodiments are possible. For example, routes of administration may include parenteral, e.g., intravenous, intradermal, subcutaneous, oral (e.g., ingestion or inhalation), transdermal (topical), transmucosal, and rectal administration. Solutions or suspensions for formulating a stem cell supertantant derived therapeutic can include: sterile diluent such as water for injection, saline solution (e.g., phosphate buffered saline (PBS, UPS)), fixed oils, glycerine, or other synthetic solvents; antibacterial and antifungal agents such as parabens, a polyol (for example, glycerol, propylene glycol, and liquid polyetheylene glycol, and the like), chlorobutanol, phenol, ascorbic acid, thimerosal, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The desired fluidity can be maintained, for example, by the use of a coating such as lecithin, by the maintenance of the required particle size in the case of dispersion and by the use of surfactants. In many cases, it will be preferable to include isotonic agents, for example, sugars, sodium chloride, polyalcohols such as mannitol or sorbitol, and in the composition. Prolonged administration of the injectable compositions can be brought about by including an agent that delays absorption. Such agents include, for example, aluminum monostearate and gelatin. The parenteral preparation can be enclosed in ampules, disposable syringes, or multiple dose vials made of glass or plastic. It is known in the art, and common practice for oral compositions to generally include an inert diluent or an edible carrier. Oral compositions can be liquid, or can be enclosed in gelatin capsules or compressed into tablets. Tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; colloidal silicon dioxide. Transmucosal administration can be accomplished through the use of nasal sprays or suppositories. For transdermal administration, the active compounds are formulated into ointments, salves, gels, or creams as generally known in the art.

Various types of cells are disclosed that are useful for the treatment of patients with valvular degeneration, however methods of therapeutically using said cells is in many cases as important in the practice of the invention as the cells themselves. One simple method of administering stem cells is through the systemic route. Although numerous studies in the area of stem cell therapy have used local administration, the high rate of blood flow has been demonstrated, at least in cardiac studies to cause systemic circulation of cells that are locally injected, with minimal retention of stem cells in the injected area. This appears to be particularly the case in balloon wire over stent approach used currently in administration of cells for post myocardial infarction setting. Systemic administration of stem cells requires dilution of cells into appropriate solutions so that cells maintain viability. In one embodiment of the invention cells are administered in a solution of phosphate buffered saline, in another embodiment cells are dissolved in a solution of saline supplemented with autologous serum at a concentration ranging between 1-10%, preferably, between 2-7%, and even more preferably at a concentration of approximately 3%. It is known to one skilled in the art that various concentrations of albumin may also be added with the saline for injection of cells. Ideally pH of the injection solution should be from about 6.4 to about 8.3, optimally 7.4. Excipients may be used to bring the solution to isotonicity such as, 4.5% mannitol or 0.9% sodium chloride, pH buffers with art-known buffer solutions. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes.

Concentration and frequency of cellular administration is dependent on patient characteristics, as well as type of stem cells used. In some situations stem cells are administered at concentrations sufficient to reduce soluble markers of inflammation such as TNF-alpha or C-reactive protein. This may be used as a marker of patient responsiveness in conditions such as mitral stenosis. The advantage of assessing anti-inflammatory activity when administering stem cell therapy is that the patient response is relatively rapid (days to weeks) when compared to other responses such as inhibition of myocardial remodeling (months to years). Numerous other factors may be used to guide the practitioner of the invention for adjusting the dose of stem cells administered. Said factors include the amount of endogenous stem cells circulating in the patient, the activity of stem cells in the patient (ie proliferative, colony formation, chemotactic mobility, etc), and the degree of valvular degeneration that is observed.

In some embodiments, stem cells are administered concomitantly with conventional medical therapy that is used for treating patients with valvular degeneration. In certain cases, medical therapy that is used, and can be used together with stem cell administration includes ACE inhibitors, beta-blockers, anticoagulants, and antihypertensives. The use of certain statins may also be performed within the context of the current invention since several such drugs have been demonstrated to actually increase the number of circulating stem cells, which correlates with therapeutic angiogenesis (145).

In other embodiments of the invention agents are administered together with stem cells in order to allow enhanced function of the administered stem cells. One such agent that is useful for systemic administration in the context of the invention is the clinically used anticonvulsant drug valproic acid. In vitro it was demonstrated that treatment of bone marrow derived hematopoietic stem cells with valproic acid increases both proliferation and self-renewal through accelerating cell cycle progression (209). Said acceleration was accompanied by a down-regulation of inhibitor factor p21(cip-1/waf-1). Furthermore, valproic acid treatment suppressed GSK3 activity and activated the Wnt signaling pathway, both of which are associated with self renewal in both hematopoietic (227, 228), but also embryonic (229, 230) stem cells. The potency of valproic acid to synergize with known hematopoietic stem cell stimulatory cytokines such as Flt3L, TPO, SCF and IL-3 was demonstrated (231). Based on this, and the known correlation between higher hematopoietic stem cell activity and myocardial protective activity, the invention teaches the use of either exogenous stem cell administration, or endogenous stem cell activity through the use of not only valproic acid, but also other known histone deacetylase inhibitors. In an embodiment of the current invention, valproic acid is administered at a concentration ranging from 20 mg/day to 1500 mg/day on a daily basis in conjunction with stem cell administration. More preferably, a dose of 150 mg/day to 1000 mg/day is given in conjunction with stem cell administration, even more preferably, a dose of 750 mg/day of valproic acid is given in conjunction with stem cell administration. One skilled in the art will understand to vary the dose based on certain characteristics of the patient, such as tolerability to valproic acid, as well as amount and rapidity of valvular regeneration/compensation that is sought. Other means of practicing the invention include administration of a compound capable of stimulating endogenous stem cell activation, proliferation, or differentiation in a manner therapeutic to valvular degeneration. One such activator of endogenous stem cells that is useful in the practice of the current invention is thalidomide.

In one embodiment of the invention, methods, compounds, and cells are disclosed that can shift the pathological process of volume or pressure overload induced myocardial hypertrophy into a physiological type of hypertrophy. It is known that in both animal models, and humans, that pregnancy induces a variety of maternal cardiac alterations, which in ways mimic volume and/or pressure induced hypertrophy. In contrast to myocardial hypertrophy associated with valvular degeneration, pregnancy associated myocardial hypertrophy is not known to be causative of pathological outcomes (146). Accordingly, through leveraging beneficial myocardial alterations that occur in pregnancy for increasing myocardial adaptation, the invention provides a novel method of treating the consequences of valvular dysfunction. In pregnancy, cardiovascular adaptation is manifested by expanded stroke volume as well as increased heart contractility. Furthermore, pregnancy triggers physiologic left ventricular hypertrophy and atrophy secondary to a transient, self-limited hemodynamic load, making the heart mechanically more efficient. One of the factors associated with cardiovascular changes is elevated female steroid hormones during gestation, which cause cardiac growth and contractility to parallel changes in various hormones, electrolyte balance, blood volume and blood pressure (147). It is known that estrogens regulate cardiac hypertrophy by direct effects on the heart and by triggering the release of cardioprotective factors such as nitric oxide synthase (NOS) and an increase of cyclic guanosine monophosphate (cGMP) (148). Myocardial eNOS appears to be important in pregnancy, where it is involved in physiological myocardial hypertrophy (149). Additionally, estrogen is known to stimulate mobilization of endothelial cells and induce inhibition of remodeling subsequent to experimental myocardial infarction (150). Accordingly, in one embodiment agents associated with pregnancy, such as estrogens, are administered prior to, in combination with, or subsequent to, stem cell administration. Concentrations of estrogen are chosen based upon the stage of myocardial compensation, and urgency of need of intervention.

In one embodiment stem cells are administered in combination with a pregnancy associated compound, or compounds known to induce ability of stem cells to self-renew and/or regenerate cardiac tissue. Said compound or compounds may be administered at a concentration that induces systemic levels similar to those observed in a pregnant woman during the highest level of cardiac hypertrophy induction. In other embodiments compounds may be administered to achieve higher or lower levels than those observed during pregnancy. On example of compounds that are useful for practicing of the current invention is human chorionic gonadotrophin (HCG) and prolactin. Administration may be daily at a concentration of 75-300 .mu.g per day, or 140 .mu.g per day for both compounds. Variations and other compounds useful for practicing the current invention are disclosed in U.S. Patent Application Publication 2006/0089309 to Joseph Tucker, hereby incorporated by reference in its entirety. Said other useful agents may include combination, or singular use of follicle-stimulating hormone (FSH), gonadotropin releasing hormone (GnRH), prolactin releasing peptide (PRP), erythropoietin, pituitary adenylate cyclase activating polypeptide (PACAP), serotonin, bone morphogenic protein (BMP), epidermal growth factor (EGF), transforming growth factor alpha (TGFalpha), transforming growth factor beta (TGFbeta), fibroblast growth factor (FGF), estrogen, growth hormone, growth hormone releasing hormone, insulin-like growth factors, leukemia inhibitory factor, ciliary neurotrophic factor (CNTF), brain derived neurotrophic factor (BDNF), thyroid hormone, thyroid stimulating hormone, and/or platelet derived growth factor (PDGF).

### EXAMPLES

### Example 1

### Treatment of Aortic Root Dilation by Systemic Administration of Cord Blood Derived Stem Cells

A patient presented with increasing diameter of the aortic root as detected by echocardiogram. On initial examination diameter of the aortic root was 47 mm. Due to the need for intervention when said aortic root radius reaches a diameter of 55 mm (ACA/AHA Guidelines for Treatment of Valvular Heart Disease (10)), said patient was treated with stem cell therapy with the aim of avoiding surgery in the short term. Umbilical cord blood was purified according to routine methods (151). Briefly, a 16-gauge needle from a standard Baxter 450-ml blood donor set containing CPD A anticoagulant (citrate/phosphate/dextrose/adenine) (Baxter Health Care, Deerfield, Ill.) was inserted and used to puncture the umbilical vein of a placenta obtained from healthy delivery from a mother tested for viral and bacterial infections according to international donor standards. Cord blood was allowed to drain by gravity so as to drip into the blood bag. The placenta was placed in a plastic-lined, absorbent cotton pad suspended from a specially constructed support frame in order to allow collection and reduce the contamination with maternal blood and other secretions, The 63 ml of CPD A used in the standard blood transfusion bag, calculated for 450 ml of blood, was reduced to 23 ml by draining 40 ml into a graduated cylinder just prior to collection. An aliquot of the cord blood was removed for safety testing according to the standards of the National Marrow Donor Program (NMDP) guidelines. Safety testing includes routine laboratory detection of human immunodeficiency virus 1 and 2, human T-cell lymphotropic virus I and II, Hepatitis B virus, Hepatitis C virus, Cytomegalovirus and Syphilis. Subsequently, 6% (wt/vol) hydroxyethyl starch was added to the anticoagulated cord blood to a final concentration of 1.2%. The leukocyte rich supernatant was then separated by centrifuging the cord blood hydroxyethyl starch mixture in the original collection blood bag (50×g for 5 min at 10° C.). The leukocyte-rich supernatant was expressed from the bag into a 150-ml Plasma Transfer bag (Baxter Health Care) and centrifuged (400×g for 10 min) to sediment the cells. Surplus supernatant plasma was transferred into a second plasma transfer bag without severing the connecting tube. Finally, the sedimented leukocytes were resuspended in supernatant plasma to a total volume of 20 ml. Approximately 5×10⁸-7×10⁹ nucleated cells were obtained per cord. Cells were cryopreserved according to the method described by Rubinstein et al (151) for subsequent cellular therapy.

Cord blood cells were washed and concentrated to a volume of 50 ml in UPS saline with 5% autologous serum. Total cell injection number was 5×10⁸ per patient. Cells were administered into systemic circulation over a period of 1 hour. The patient was observed for adverse events for the first 12 hours subsequent to administration and subsequently discharged. No adverse events were observed associated with the procedure or in the follow up. A second echocardiogram was performed using identical parameters as the initial one. Patient aortic root diameter was observed to decrease to 43 mm. This observation is particularly stunning since aortic root dilation is a progressive disease of increasing diameter, and to the knowledge of the inventors, no case has been reported of spontaneous aortic root dilation reversion.

### Example 2

### Autologous Bone Marrow Derived Stem Cell Therapy for Aortic Insufficiency

50 patients suffering from asymptomatic severe aortic regurgitation and normal left ventricular function are assigned to the stem cell treatment group, whereas 50 patients with similar conditions are assigned to the placebo group. Aortic regurgitation is quantified as severe when the jet width exceeded 10 mm and the apical jet area exceeded 7 cm2 on color Doppler ultrasonography, or when the regurgitant fraction exceeds 60% Patients with any of the following characteristics are excluded from the study: a decreased left ventricular ejection fraction (less than 50 percent) during the preceding six months, other clinically significant associated valvular disease, associated valvular aortic stenosis (aortic mean gradient, more than 20 mm Hg), a diastolic blood pressure of more than 90 mm Hg, or atrial fibrillation.

Patients in the treatment group are subjected to a bone marrow harvest. Briefly, patients are positioned face down on a horizontal platform and provided analgesics as per standard medical procedures. All personnel involved in the procedure are dressed in sterile surgical gowning and masks. The harvesting field consisting of both iliac crests is prepared by topically applying standard disinfectant solution. Iliac crests are anaesthetized and the harvesting needle is inserted in order to puncture the iliac crest. The cap and stylet of the harvesting needle is removed and 3-ml of marrow is harvested into the 15-ml harvesting syringe containing heparin solution. The process is repeated and the contents of the harvesting syringe are transferred into a 500-ml collecting bag. Approximately 75-125 ml of bone marrow is harvested in total.

Isolation of mononuclear cells is performed by gradient separation using the Hetastarch method, which is clinically applicable and reported to remove not only erythrocytes but also granulocytic cells. The previously published method of Montuoro et al is used (152). Briefly, six-percent (wt/vol) Hetastarch (HES40, Hishiyama Pharmaceutical Co., Osaka, Japan) is added to the collected bone marrow sample to achieve a final concentration of 1.2 percent Hetastarch, (1:5 volume ratio of added Hetastarch to bone marrow). Centrifugation at 50 g for 5 min at 10° C. is performed in order to generate a leukocyte rich supernatant. Sedimentation of bone marrow takes place at a cell concentration of no more than 15×10⁶ cells/ml in a total volume of 850 ml per Hetastarch bag. The supernatant is transferred into a plasma transfer bag and centrifuged (400 g for 10 min) to sediment the cells. The sedimented cells are subsequently washed in phosphate buffered saline in the presence of 5% penicillin/streptomycin mixture (Gibco, Mississauga, Canada) and 5% autologous serum. Cellular viability and lack of potential contamination with other cells is assessed by microscopy. Bone marrow mononuclear cells are subsequently concentrated.

Cells are washed and concentrated to a volume of 50 ml in UPS saline with 5% autologous serum. Total cell injection number is approximately 5×10⁸ per patient. Cells are administered into systemic circulation over a period of 1 hour. Patient are observed for adverse events for the first 12 hours subsequent to administration and subsequently discharged. No adverse events are observed associated with the procedure or in the follow up. The procedure is repeated once every 6 months in patients in the treatment group. At 2-year evaluation 2 patients from the treated group require valve replacement surgery according to ACA/AHA guidelines, whereas 13 patients from the control group require surgery.

### Example 3

### Systemic Administration of Cord Blood Derived Stem Cells in Combination with Histone Deacetylase Inhibitor Therapy for Aortic Regurgitation Induced Left Ventricular Remodeling

A clinical trial is performed in a total of 200 patients suffering from eccentric hypertrophy as a result of aortic regurgitation. 50 patients are placed in the placebo group (Group 1) 50 in the stem cells alone treatment group (Group 2), 50 in the valproic acid alone group (Group 3), and 50 in the combination group of stem cell therapy and valproic acid administration group (Group 4). All patients entering the study have progressive enlargement of the left ventricle as determined by 2 consecutive echocardiograms. To be eligible to participate, patients must have a LVEF>than 55% at study entrance. The average end diastolic left ventricle diameter at study onset is: Group 1 55.3 mm; Group 2 55.9 mm; Group 3 54 mm; and Group 4 56 mm. Unmatched umbilical cord blood stem cell therapy is performed as described in Example 1 in patients randomized to the stem cells alone and stem cells plus valproic acid groups. Valproic acid is administered orally for 2 weeks before stem cell administration in the relevant group. Administration of valproic acid is performed orally at a concentration of 750 mg/day. Patients are followed for the period of 1 year, with valproic acid administration occurring daily for said period. At 1-year follow-up the average left ventricle systolic diameter is 75 mm for patients in Group 1, 62 mm for patients in Group 2, 68 mm for patients in Group 3, and 51 mm for patients in Group 4.

### Example 4

### Administration of Umbilical Cord Blood Derived Mesenchymal Stem Cell for Mitral Stenosis-Induced Pressure Remodeling

20 patients with advanced mitral stenosis and atrial fibrillation who are ineligible for percutaneous or surgical intervention according to the ACC/AHA guidelines are treated by systemic administration of cord blood derived mesenchymal stem cells with the goal of inducing therapeutic tissue remodeling by inhibiting fibrosis, thus allowing proper opening of the mitral oriface. At presentation the mean area of the mitral valve orifice is 1.4 cm² for all patients. Additionally, the average pressure gradient of the patients across the mitral valve is 11 mm Hg. Cord blood is obtained, processed and HLA-matched according to EXAMPLE 1. Cord blood mononuclear cells are seeded at a density of 1×10⁶ cells/cm² into culture flasks in a Good Manufacturing Procedures-compliant sterile clean room. Cells are cultured in DMEM-LG media (Life Technologies), supplemented with 10% autologous serum. On day 4, nonadherent cells are discarded and fresh tissue culture medium is added. On day 7, cultures are tested for sterility, nonadherent cells were discarded by washing culture flasks with USP saline containing 10% autologous serum, and the remaining adherent cells are washed with Tyrode's Salt Solution (Sigma, St. Louis, Mo.) and incubated for 1 hr in M199 mediua (Life Technologies). Cells are detached with 0.05% trypsin-EDTA (Life Technologies), and are resuspended in M199 supplemented with 10% of autologous serum. Cells are subcultured for an 12 days with feeding of cultures performed every 3 days. The cells are subsequently harvested by trypsinization as described above, counted and an aliquot is taken for flow cytometric analyzes for the expression of mesenchymal stem cells markers and lack of expression of hematopoietic markers. Cell batches of >95% purity for CD73, and CD105, and less than 5% contamination of CD45 expressing cells are chosen for cell therapy. Cell concentration is adjusted to 5×10⁷ cells in USP saline supplemented with 10% autologous serum and injected systemically in a volume of 50 ml in the period of 2 hours. Injection is performed once every two weeks for a total of 4 injections. Patients are maintained on standard medical therapy. At 6-month evaluation the mean area of the mitral valve orifice in patients is 3.3 cm². Additionally, the average pressure gradient of the patients across the mitral valve is reduced to 5.9 mm Hg.

### Example 5

### Treating Mitral Regurgitation Through Oral Administration of a Stem Cell Activator

10 patients presenting with ischemia associated mitral regurgitation with an overall average LVEF of 53.4%, and a regurgitation fraction of 35% on average. Said patients are treated with thalidomide 100 mg/day for four months. Subsequent to treatment, LVEF is re-assessed using identical echocardiogram methodology. Average LVEF is increased to 74%, and interestingly the regurgitant fraction is decreased on average to 22.4%. One patient died of causes unrelated to the intervention. Autopsy findings reveal an expanded number of proliferating (PCNA+) CD34+ cells in both the posterior and anterior mitral valves.

### Example 6

### Treating Mitral Prolapse Through Systemic Stem Cell Administration

20 patients with flail valve mitral prolapse are treated by administration of cord blood stem cells as described in Example 1. Subsequent to treatment heart murmur is decreased in severity in 7 of the patients and 8 patients with NYHA class II heart failure revert to NYHA class I failure.

One skilled in the art will appreciate that these methods, compositions, and cells are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. It will be apparent to one skilled in the art that varying substitutions and modifications may be made to the invention disclosed herein without departing from the scope and spirit of the invention. Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions indicates the exclusion of equivalents of the features shown and described or portions thereof. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

### REFERENCES

The following references are each incorporated herein by reference in their entirety
1. Donal, E., Levy, F., and Tribouilloy, C. 2006. Chronic ischemic mitral regurgitation. J Heart Valve Dis 15:149-157.
2. Rose, A. G. 1996. Etiology of valvular heart disease. Curr Opin Cardiol 11:98-113.
3. Cotrufo, M., Agozzino, L., De Feo, M., Della Corte, A., De Santo, L. S., Di Benedetto, G., Esposito, S., and Nappi, G. 2003. Aortic valve dysfunction and dilated ascending aorta. A complex and controversial association. Ital Heart J 4:589-595.
4. Galioto, F. M., Jr., Midgley, F. M., Shapiro, S. R., Perry, L. W., Ciaravella, J. M., Jr., and Scott, L. P., 3rd. 1981. Mitral valve replacement in infants and children. Pediatrics 67:230-235.
5. Nasuti, J. F., Zhang, P. J., Feldman, M. D., Pasha, T., Khurana, J. S., Gorman, J. H., 3rd, Gorman, R. C., Narula, J., and Narula, N. 2004. Fibrillin and other matrix proteins in mitral valve prolapse syndrome. Ann Thorac Surg 77:532-536.
6. Devereux, R. B., Kramer-Fox, R., Shear, M. K., Kligfield, P., Pini, R., and Savage, D. D. 1987. Diagnosis and classification of severity of mitral valve prolapse: methodologic, biologic, and prognostic considerations. Am Heart J 113:1265-1280.
7. Ortega Gil, J., and Rodriguez Castro, J. M. 2005. Mitral valvular heart disease in the adults. Bol Asoc Med P R 97:283-293.
8. Essop, M. R., and Nkomo, V. T. 2005. Rheumatic and nonrheumatic valvular heart disease: epidemiology, management, and prevention in Africa. Circulation 112:3584-3591.
9. Iskandrian, A. S., Hakki, A. H., and Kotler, M. N. 1986. Value of echocardiography and nuclear techniques in assessment of valvular heart lesions. Cardiovasc Clin 17:181-200.
10. Bonow, R. O., Carabello, B. A., Kanu, C., de Leon, A. C., Jr., Faxon, D. P., Freed, M. D., Gaasch, W. H., Lytle, B. W., Nishimura, R. A., O'Gara, P. T., et al. 2006. ACC/AHA 2006 guidelines for the management of patients with valvular heart disease: a report of the American College of Cardiology/American Heart Association Task Force on Practice Guidelines (writing committee to revise the 1998 Guidelines for the Management of Patients With Valvular Heart Disease): developed in collaboration with the Society of Cardiovascular Anesthesiologists: endorsed by the Society for Cardiovascular Angiography and Interventions and the Society of Thoracic Surgeons. Circulation 114:e84-231.
11. Jonkaitiene, R., Benetis, R., Ablonskyte-Dudoniene, R., and Jurkevicius, R. 2005. Mitral valve prolapse: diagnosis, treatment and natural course. Medicina (Kaunas) 41:325-334.
12. Lawrie, G. M. 1998. Mitral valve repair vs replacement. Current recommendations and long-term results. Cardiol Clin 16:437-448.
13. Opie, L. H., Commerford, P. J., Gersh, B. J., and Pfeffer, M. A. 2006. Controversies in ventricular remodelling. Lancet 367:356-367.
14. Robin, J., Ninet, J., Jarolin, G., Dugres, B., Chassignolle, J. F., and Champsaur, G. 1994. [Post-infarction rupture of the heart into the pericardium. Surgical treatment combining synthetic patch and biological fibrin glue]. Presse Med 23:1435-1438.
15. Kelbaek, H., Aldershvile, J., Skagen, K., Hildebrandt, P., and Nielsen, S. L. 1996. Pre- and afterload reduction in chronic mitral regurgitation: a double-blind randomized placebo-controlled trial of the acute and 2 weeks' effect of nifedipine or isosorbide dinitrate treatment on left ventricular function and the severity of mitral regurgitation. Br J Clin Pharmacol 41:493-497.
16. 1992. Improved diastolic function with the calcium antagonist nisoldipine (coat-core) in patients post myocardial infarction: results of the DEFIANT study. Doppler Flow and Echocardiography in Functional cardiac Insufficiency: Assessment of Nisoldipine Therapy. Eur Heart J 13:1496-1505.
17. Carabello, B. A. 2005. Modern management of mitral stenosis. Circulation 112:432-437.
18. Moreyra, A. E., Wilson, A. C., Deac, R., Suciu, C., Kostis, J. B., Ortan, F., Kovacs, T., and Mahalingham, B. 1998. Factors associated with atrial fibrillation in patients with mitral stenosis: a cardiac catheterization study. Am Heart J 135:138-145.
19. Boon, N. A., and Bloomfield, P. 2002. The medical management of valvar heart disease. Heart 87:395-400.
20. Maurer, G. 2006. Aortic regurgitation. Heart 92:994-1000.
21. Zeppilli, P., Bianco, M., Bria, S., and Palmieri, V. 2006. Bicuspid aortic valve: an innocent finding or a potentially life-threatening anomaly whose complications may be elicited by sports activity? J Cardiovasc Med (Hagerstown) 7:282-287.
22. Rosenhek, R., Rader, F., Klaar, U., Gabriel, H., Krejc, M., Kalbeck, D., Schemper, M., Maurer, G., and Baumgartner, H. 2006. Outcome of watchful waiting in asymptomatic severe mitral regurgitation. Circulation 113:2238-2244.
23. Pengo, V., Barbero, F., Biasiolo, A., Pegoraro, C., Noventa, F., and Iliceto, S. 2003. Prevention of thromboembolism in patients with mitral stenosis and associated atrial fibrillation: effectiveness of low intensity (INR target 2) oral anticoagulant treatment. Thromb Haemost 89:760-764.
24. Roldan, C. A., Gelgand, E. A., Quails, C. R., and Sibbitt, W. L., Jr. 2005. Valvular heart disease as a cause of cerebrovascular disease in patients with systemic lupus erythematosus. Am J Cardiol 95:1441-1447.
25. Gerasimou, A., Oliaro, E., Quaglia, C., Canta, M., Calachanis, M., Morello, M., and Mangiardi, L. 2004. When should asymptomatic patients with combined severe aortic stenosis and aortic insufficiency undergo valve replacement? A clinical case. Minerva Cardioangiol 52:339-344.
26. Deantonio, H. J., and Movahed, A. 1992. Atrial fibrillation: current therapeutic approaches. Am Fam Physician 45:2576-2584.
27. Demellis, J. M., and Panaretou, M. P. 2003. Effects of digoxin on left atrial function in heart failure. Heart 89:1308-1315.
28. Chan, W. L., Gilligan, D. M., Ang, E. L., and Oakley, C. M. 1990. Effect of preload change on resting and exercise cardiac performance in hypertrophic cardiomyopathy. Am J Cardiol 66:746-751.
29. Levine, H. J., and Gaasch, W. H. 1996. Vasoactive drugs in chronic regurgitant lesions of the mitral and aortic valves. J Am Coll Cardiol 28:1083-1091.
30. Feldman, T., Wasserman, H. S., Herrmann, H. C., Gray, W., Block, P. C., Whitlow, P., St Goar, F., Rodriguez, L., Silvestry, F., Schwartz, A., et al. 2005. Percutaneous mitral valve repair using the edge-to-edge technique: six-month results of the EVEREST Phase I Clinical Trial. J Am Coll Cardiol 46:2134-2140.
31. Webb, J. G., Chandavimol, M., Thompson, C. R., Ricci, D. R., Carere, R. G., Munt, B. I., Buller, C. E., Pasupati, S., and Lichtenstein, S. 2006. Percutaneous aortic valve implantation retrograde from the femoral artery. Circulation 113:842-850.
32. Webb, J. G., Harnek, J., Munt, B. I., Kimblad, P. O., Chandavimol, M., Thompson, C. R., Mayo, J. R., and Solem, J. O. 2006. Percutaneous transvenous mitral annuloplasty: initial human experience with device implantation in the coronary sinus. Circulation 113:851-855.
33. Khambadkone, S., Coats, L., Taylor, A., Boudjemline, Y., Derrick, G., Tsang, V., Cooper, J., Muthurangu, V., Hegde, S. R., Razavi, R. S., et al. 2005. Percutaneous pulmonary valve implantation in humans: results in 59 consecutive patients. Circulation 112:1189-1197.
34. Wisenbaugh, T. 1994. Mitral valve disease. Curr Opin Cardiol 9:146-151.
35. Kennedy, J. W., Doces, J., and Stewart, D. K. 1977. Left ventricular function before and following aortic valve replacement. Circulation 56:944-950.
36. de Sa, M., Moshkovitz, Y., Butany, J., and David, T. E. 1999. Histologic abnormalities of the ascending aorta and pulmonary trunk in patients with bicuspid aortic valve disease: clinical relevance to the ross procedure. J Thorac Cardiovasc Surg 118:588-594.
37. Halme, T., Savunen, T., Aho, H., Vihersaari, T., and Penttinen, R. 1985. Elastin and collagen in the aortic wall: changes in the Marfan syndrome and annuloaortic ectasia. Exp Mol Pathol 43:1-12.
38. Karakaya, O., Barutcu, I., Esen, A. M., Dogan, S., Saglam, M., Karapinar, H., Akgun, T., Karavelioglu, Y., Esen, O., Ozdemir, N., et al. 2006. Relationship between circulating plasma matrix metalloproteinase-9 (gelatinase-B) concentration and aortic root dilatation. Am J Hypertens 19:361-365.
39. Ninichuk, V., Gross, O., Segerer, S., Hoffmann, R., Radomska, E., Buchstaller, A., Huss, R., Akis, N., Schlondorff, D., and Anders, H. J. 2006. Multipotent mesenchymal stem cells reduce interstitial fibrosis but do not delay progression of chronic kidney disease in collagen4A3-deficient mice. Kidney Int 70:121-129.
40. Zhao, D. C., Lei, J. X., Chen, R., Yu, W. H., Zhang, X. M., Li, S. N., and Xiang, P. 2005. Bone marrow-derived mesenchymal stem cells protect against experimental liver fibrosis in rats. World J Gastroenterol 11:3431-3440.
41. Gomperts, B. N., and Strieter, R. M. 2006. Stem Cells and Chronic Lung Disease. Annu Rev Med.
42. Guarita-Souza, L. C., Carvalho, K. A., Woitowicz, V., Rebelatto, C., Senegaglia, A., Hansen, P., Miyague, N., Francisco, J. C., Olandoski, M., Faria-Neto, J. R., et al. 2006. Simultaneous autologous transplantation of cocultured mesenchymal stem cells and skeletal myoblasts improves ventricular function in a murine model of chagas disease. Circulation 114:1120-124.
43. Caplan, A. I., and Dennis, J. E. 2006. Mesenchymal stem cells as trophic mediators. J Cell Biochem.
44. Fazel, S., Cimini, M., Chen, L., Li, S., Angoulvant, D., Fedak, P., Verma, S., Weisel, R. D., Keating, A., and Li, R. K. 2006. Cardioprotective c-kit+ cells are from the bone marrow and regulate the myocardial balance of angiogenic cytokines. J Clin Invest 116:1865-1877.
45. Fox, C. S., Guo, C. Y., Larson, M. G., Vasan, R. S., Parise, H., O'Donnell, C. J., D'Agostino, R. B., Sr., Keaney, J. F., Jr., and Benjamin, E. J. 2006. Relations of inflammation and novel risk factors to valvular calcification. Am J Cardiol 97:1502-1505.
46. Uccelli, A., Zappia, E., Benvenuto, F., Frassoni, F., and Mancardi, G. 2006. Stem cells in inflammatory demyelinating disorders: a dual role for immunosuppression and neuroprotection. Expert Opin Biol Ther 6:17-22.
47. Berry, M. F., Engler, A. J., Woo, Y. J., Pirolli, T. J., Bish, L. T., Jayasankar, V., Morine, K. J., Gardner, T. J., Discher, D. E., and Sweeney, H. L. 2006. Mesenchymal stem cell injection after myocardial infarction improves myocardial compliance. Am J Physiol Heart Circ Physiol 290:H2196-2203.
48. Silva, G. V., Litovsky, S., Assad, J. A., Sousa, A. L., Martin, B. J., Vela, D., Coulter, S. C., Lin, J., Ober, J., Vaughn, W. K., et al. 2005. Mesenchymal stem cells differentiate into an endothelial phenotype, enhance vascular density, and improve heart function in a canine chronic ischemia model. Circulation 111:150-156.
49. Shah, R. V., and Mitchell, R. N. 2005. The role of stem cells in the response to myocardial and vascular wall injury. Cardiovasc Pathol 14:225-231.
50. Uemura, R., Xu, M., Ahmad, N., and Ashraf, M. 2006. Bone marrow stem cells prevent left ventricular remodeling of ischemic heart through paracrine signaling. Circ Res 98:1414-1421.
51. Lee, N., Thorne, T., Losordo, D. W., and Yoon, Y. S. 2005. Repair of ischemic heart disease with novel bone marrow-derived multipotent stem cells. Cell Cycle 4:861-864.
52. Aukrust, P., Yndestad, A., Damas, J. K., and Gullestad, L. 2004. Inflammation and chronic heart failure-potential therapeutic role of intravenous immunoglobulin. Autoimmun Rev 3:221-227.
53. Aggarwal, S., and Pittenger, M. F. 2005. Human mesenchymal stem cells modulate allogeneic immune cell responses. Blood 105:1815-1822.
54. Marinova-Mutafchieva, L., Williams, R. O., Funa, K., Maini, R. N., and Zvaifler, N. J. 2002. Inflammation is preceded by tumor necrosis factor-dependent infiltration of mesenchymal cells in experimental arthritis. Arthritis Rheum 46:507-513.
55. Hou, M., Yang, K. M., Zhang, H., Zhu, W. Q., Duan, F. J., Wang, H., Song, Y. H., Wei, Y. J., and Hu, S. S. 2006. Transplantation of mesenchymal stem cells from human bone marrow improves damaged heart function in rats. Int J. Cardiol.
56. Yang, Z. J., Ma, D. C., Wang, W., Xu, S. L., Zhang, Y. Q., Chen, B., Zhou, F., Zhu, T. B., Wang, L. S., Xu, Z. Q., et al. 2006. Experimental study of bone marrow-derived mesenchymal stem cells combined with hepatocyte growth factor transplantation via noninfarct-relative artery in acute myocardial infarction. Gene Ther.
57. Ge, J., Li, Y., Qian, J., Shi, J., Wang, Q., Niu, Y., Fan, B. P., Liu, X., Zhang, S., Sun, A., et al. 2006. Efficacy of Emergent Transcatheter Transplantation of Stem Cells for Treatment of Acute Myocardial Infarction (TCT-STAMI). Heart.
58. Musialek, P., Tracz, W., Skotnicki, A. B., Zmudka, K., Pieniazek, P., Walter, Z., Szostek, M., Majka, M., Weglarska, D., Zalewski, J., et al. 2006. Transcoronary stem cell delivery using physiological endothelium-targeting perfusion technique: the rationale and a pilot study involving a comparison with conventional over-the-wire balloon coronary occlusions in patients after recent myocardial infarction. Kardiol Pol 64:489-498; discussion 499.
59. Anversa, P., Leri, A., and Kajstura, J. 2006. Cardiac regeneration. J Am Coll Cardiol 47:1769-1776.
60. Nagaya, N., Kangawa, K., Itoh, T., Iwase, T., Murakami, S., Miyahara, Y., Fujii, T., Uematsu, M., Ohgushi, H., Yamagishi, M., et al. 2005. Transplantation of mesenchymal stem cells improves cardiac function in a rat model of dilated cardiomyopathy. Circulation 112:1128-1135.
61. Wang, J. A., Xie, X. J., He, H., Sun, Y., Jiang, J., Luo, R. H., Fan, Y. Q., and Dong, L. 2006. [A prospective, randomized, controlled trial of autologous mesenchymal stem cells transplantation for dilated cardiomyopathy]. Zhonghua Xin Xue Guan Bing Za Zhi 34:107-110.
62. Bokeriia, L. A., Buziashvili Iu, I., Matskeplishvili, S. T., and Kamardinov, D. 2004. [First experience with the use of bone marrow stem cells for regeneration therapy of ischemic heart disease.]. Kardiologiia 44:16-22.
63. Patel, A. N., Geffner, L., Vina, R. F., Saslavsky, J., Urschel, H. C., Jr., Kormos, R., and Benetti, F. 2005. Surgical treatment for congestive heart failure with autologous adult stem cell transplantation: a prospective randomized study. J Thorac Cardiovasc Surg 130:1631-1638.
64. Sutherland, F. W., Perry, T. E., Yu, Y., Sherwood, M. C., Rabkin, E., Masuda, Y., Garcia, G. A., McLellan, D. L., Engelmayr, G. C., Jr., Sacks, M. S., et al. 2005. From stem cells to viable autologous semilunar heart valve. Circulation 111:2783-2791.
65. Ku, C. H., Johnson, P. H., Batten, P., Sarathchandra, P., Chambers, R. C., Taylor, P. M., Yacoub, M. H., and Chester, A. H. 2006. Collagen synthesis by mesenchymal stem cells and aortic valve interstitial cells in response to mechanical stretch. Cardiovasc Res 71:548-556.
66. Rajamannan, N. M., Subramaniam, M., Rickard, D., Stock, S. R., Donovan, J., Springett, M., Orszulak, T., Fullerton, D. A., Tajik, A. J., Bonow, R. O., et al. 2003. Human aortic valve calcification is associated with an osteoblast phenotype. Circulation 107:2181-2184.
67. Higashi, Y., Nishioka, K., Umemura, T., Chayama, K., and Yoshizumi, M. 2006. Oxidative stress, endothelial function and angiogenesis induced by cell therapy and gene therapy. Curr Pharm Biotechnol 7:109-116.
68. Tonnesen, M. G., Feng, X., and Clark, R. A. 2000. Angiogenesis in wound healing. J Investig Dermatol Symp Proc 5:40-46.
69. Chen, M. C., Chang, H. W., Wu, C. J., Yang, C. H., Yu, T. H., Chen, C. J., and Hung, W. C. 2005. Balance between plasma levels of tumor necrosis factor-alpha and interleukin-10 in rheumatic mitral stenosis. Cardiology 104:171-175.
70. Bond, M., Fabunmi, R. P., Baker, A. H., and Newby, A. C. 1998. Synergistic upregulation of metalloproteinase-9 by growth factors and inflammatory cytokines: an absolute requirement for transcription factor NF-kappa B. FEBS Lett 435:29-34.
71. Zimmermann, O., Kochs, M., Zwaka, T. P., Bienek-Ziolkowski, M., Hoher, M., Hombach, V., and Torzewski, J. 2006. Prognostic role of myocardial tumor necrosis factor-alpha and terminal complement complex expression in patients with dilated cardiomyopathy. Eur J Heart Fail.
72. Kaur, K., Sharma, A. K., and Singal, P. K. 2006. Significance of changes in TNF-alpha and IL-10 levels in the progression of heart failure subsequent to myocardial infarction. Am J Physiol Heart Circ Physiol 291:H106-113.
73. Bekeredjian, R., and Grayburn, P. A. 2005. Valvular heart disease: aortic regurgitation. Circulation 112:125-134.
74. Rubinstein, P., Carrier, C., Scaradavou, A., Kurtzberg, J., Adamson, J., Migliaccio, A. R., Berkowitz, R. L., Cabbad, M., Dobrila, N. L., Taylor, P. E., et al. 1998. Outcomes among 562 recipients of placental-blood transplants from unrelated donors. N Engl J Med 339:1565-1577.
75. Sirchia, G., and Rebulla, P. 1999. Placental/umbilical cord blood transplantation. Haematologica 84:738-747.
76. Demir, R., Kosanke, G., Kohnen, G., Kertschanska, S., and Kaufmann, P. 1997. Classification of human placental stem villi: review of structural and functional aspects. Microsc Res Tech 38:29-41.
77. Portmann-Lanz, C. B., Schoeberlein, A., Huber, A., Sager, R., Malek, A., Holzgreve, W., and Surbek, D. V. 2006. Placental mesenchymal stem cells as potential autologous graft for pre- and perinatal neuroregeneration. Am J Obstet Gynecol 194:664-673.
78. Mohamed, A. A., Ibrahim, A. M., El-Masry, M. W., Mansour, I. M., Khroshied, M. A., Gouda, H. M., and Riad, R. M. 2006. Ex vivo expansion of stem cells: defining optimum conditions using various cytokines. Lab Hematol 12:86-93.
79. Kashiwakura, I., and Takahashi, T. A. 2005. Fibroblast growth factor and ex vivo expansion of hematopoietic progenitor cells. Leuk Lymphoma 46:329-333.
80. Mineishi, S. 2003. [Immunobiology of mini-transplant]. Nippon Rinsho 61:1489-1494.
81. Bossolasco, P., Montemurro, T., Cova, L., Zangrossi, S., Calzarossa, C., Buiatiotis, S., Soligo, D., Bosari, S., Silani, V., Deliliers, G. L., et al. 2006. Molecular and phenotypic characterization of human amniotic fluid cells and their differentiation potential. Cell Res 16:329-336.
82. Sartore, S., Lenzi, M., Angelini, A., Chiavegato, A., Gasparotto, L., De Coppi, P., Bianco, R., and Gerosa, G. 2005. Amniotic mesenchymal cells autotransplanted in a porcine model of cardiac ischemia do not differentiate to cardiogenic phenotypes. Eur J Cardiothorac Surg 28:677-684.
83. Prusa, A. R., Marton, E., Rosner, M., Bettelheim, D., Lubec, G., Pollack, A., Bernaschek, G., and Hengstschlager, M. 2004. Neurogenic cells in human amniotic fluid. Am J Obstet Gynecol 191:309-314.
84. Tsai, M. S., Hwang, S. M., Tsai, Y. L., Cheng, F. C., Lee, J. L., and Chang, Y. J. 2006. Clonal amniotic fluid-derived stem cells express characteristics of both mesenchymal and neural stem cells. Biol Reprod 74:545-551.
85. Lalu, M. M., Pasini, E., Schulze, C. J., Ferrari-Vivaldi, M., Ferrari-Vivaldi, G., Bachetti, T., and Schulz, R. 2005. Ischaemia-reperfusion injury activates matrix metalloproteinases in the human heart. Eur Heart J 26:27-35.
86. Kameda, K., Matsunaga, T., Abe, N., Hanada, H., Ishizaka, H., Ono, H., Saitoh, M., Fukui, K., Fukuda, I., Osanai, T., et al. 2003. Correlation of oxidative stress with activity of matrix metalloproteinase in patients with coronary artery disease. Possible role for left ventricular remodelling. Eur Heart J 24:2180-2185.
87. Reinhardt, D., Sigusch, H. H., Hensse, J., Tyagi, S. C., Korfer, R., and Figulla, H. R. 2002. Cardiac remodelling in end stage heart failure: upregulation of matrix metalloproteinase (MMP) irrespective of the underlying disease, and evidence for a direct inhibitory effect of ACE inhibitors on MMP. Heart 88:525-530.
88. Wilson, E. M., and Spinale, F. G. 2001. Myocardial remodelling and matrix metalloproteinases in heart failure: turmoil within the interstitium. Ann Med 33:623-634.
89. Tamura, M., Unno, K., Yonezawa, S., Hattori, K., Nakashima, E., Tsukada, H., Nakajima, M., and Oku, N. 2004. In vivo trafficking of endothelial progenitor cells their possible involvement in the tumor neovascularization. Life Sci 75:575-584.
90. Werner, N., Kosiol, S., Schiegl, T., Ahlers, P., Walenta, K., Link, A., Bohm, M., and Nickenig, G. 2005. Circulating endothelial progenitor cells and cardiovascular outcomes. N Engl J Med 353:999-1007.
91. Wojakowski, W., and Tendera, M. 2005. Mobilization of bone marrow-derived progenitor cells in acute coronary syndromes. Folia Histochem Cytobiol 43:229-232.
92. Hristov, M., and Weber, C. 2004. Endothelial progenitor cells: characterization, pathophysiology, and possible clinical relevance. J Cell Mol Med 8:498-508.
93. Laufs, U., Urhausen, A., Werner, N., Scharhag, J., Heitz, A., Kissner, G., Bohm, M., Kindermann, W., and Nickenig, G. 2005. Running exercise of different duration and intensity: effect on endothelial progenitor cells in healthy subjects. Eur J Cardiovasc Prev Rehabil 12:407-414.
94. Knippenberg, M., Helder, M. N., Doulabi, B. Z., Semeins, C. M., Wuisman, P. I., and Klein-Nulend, J. 2005. Adipose tissue-derived mesenchymal stem cells acquire bone cell-like responsiveness to fluid shear stress on osteogenic stimulation. Tissue Eng 11:1780-1788.
95. Zhang, X., Mitsuru, A., Igura, K., Takahashi, K., Ichinose, S., Yamaguchi, S., and Takahashi, T. A. 2006. Mesenchymal progenitor cells derived from chorionic villi of human placenta for cartilage tissue engineering. Biochem Biophys Res Commun 340:944-952.
96. Shih, D. T., Lee, D. C., Chen, S. C., Tsai, R. Y., Huang, C. T., Tsai, C. C., Shen, E. Y., and Chiu, W. T. 2005. Isolation and characterization of neurogenic mesenchymal stem cells in human scalp tissue. Stem Cells 23:1012-1020.
97. Kadivar, M., Khatami, S., Mortazavi, Y., Shokrgozar, M. A., Taghikhani, M., and Soleimani, M. 2006. In vitro cardiomyogenic potential of human umbilical vein-derived mesenchymal stem cells. Biochem Biophys Res Commun 340:639-647.
98. Kern, S., Eichler, H., Stoeve, J., Kluter, H., and Bieback, K. 2006. Comparative Analysis of Mesenchymal Stem Cells from Bone Marrow, Umbilical Cord Blood or Adipose Tissue. Stem Cells.
99. Jansen, J. H., Fibbe, W. E., Willemze, R., and Kluin-Nelemans, J. C. 1990. Interleukin-4. A regulatory protein. Blut 60:269-274.
100. Zhou, X., Schmidtke, P., Zepp, F., and Meyer, C. U. 2005. Boosting interleukin-10 production: therapeutic effects and mechanisms. Curr Drug Targets Immune Endocr Metabol Disord 5:465-475.
101. Mentink-Kane, M. M., and Wynn, T. A. 2004. Opposing roles for IL-13 and IL-13 receptor alpha 2 in health and disease. Immunol Rev 202:191-202.
102. Kriegel, M. A., Li, M. O., Sanjabi, S., Wan, Y. Y., and Flavell, R. A. 2006. Transforming growth factor-beta: recent advances on its role in immune tolerance. Curr Rheumatol Rep 8:138-144.
103. Fernandez-Botran, R., Crespo, F. A., and Sun, X. 2002. Soluble cytokine receptors in biological therapy. Expert Opin Biol Ther 2:585-605.
104. Dayer, J. M. 2002. Evidence for the biological modulation of IL-1 activity: the role of IL-1Ra. Clin Exp Rheumatol 20:S14-20.
105. Rouas-Freiss, N., Moreau, P., Ferrone, S., and Carosella, E. D. 2005. HLA-G proteins in cancer: do they provide tumor cells with an escape mechanism? Cancer Res 65:10139-10144.
106. Bohana-Kashtan, O., and Civin, C. I. 2004. Fas ligand as a tool for immunosuppression and generation of immune tolerance. Stem Cells 22:908-924.
107. Okazaki, T., and Honjo, T. 2006. The PD-1-PD-L pathway in immunological tolerance. Trends Immunol 27:195-201.
108. Longhi, M. P., Harris, C. L., Morgan, B. P., and Gallimore, A. 2006. Holding T cells in check—a new role for complement regulators? Trends Immunol 27:102-108.
109. Wahl, S. M., Orenstein, J. M., and Chen, W. 2000. TGF-beta influences the life and death decisions of T lymphocytes. Cytokine Growth Factor Rev 11:71-79.
110. Mellor, A. L., and Munn, D. H. 2004. IDO expression by dendritic cells: tolerance and tryptophan catabolism. Nat Rev Immunol 4:762-774.
111. Serafini, P., Borrello, I., and Bronte, V. 2006. Myeloid suppressor cells in cancer: recruitment, phenotype, properties, and mechanisms of immune suppression. Semin Cancer Biol 16:53-65.
112. Matsumoto, R., Omura, T., Yoshiyama, M., Hayashi, T., Inamoto, S., Koh, K. R., Ohta, K., Izumi, Y., Nakamura, Y., Akioka, K., et al. 2005. Vascular endothelial growth factor-expressing mesenchymal stem cell transplantation for the treatment of acute myocardial infarction. Arterioscler Thromb Vasc Biol 25:1168-1173.
113. Klein, S., Roghani, M., and Rifkin, D. B. 1997. Fibroblast growth factors as angiogenesis factors: new insights into their mechanism of action. Exs 79:159-192.
114. Chen, C. H., Poucher, S. M., Lu, J., and Henry, P. D. 2004. Fibroblast growth factor 2: from laboratory evidence to clinical application. Curr Vasc Pharmacol 2:33-43.
115. Grines, C., Rubanyi, G. M., Kleiman, N. S., Marrott, P., and Watkins, M. W. 2003. Angiogenic gene therapy with adenovirus 5 fibroblast growth factor-4 (Ad5FGF-4): a new option for the treatment of coronary artery disease. Am J Cardiol 92:24 N-31N.
116. Dufourcq, P., Couffinhal, T., Ezan, J., Barandon, L., Moreau, C., Daret, D., and Duplaa, C. 2002. FrzA, a secreted frizzled related protein, induced angiogenic response. Circulation 106:3097-3103.
117. Morisada, T., Kubota, Y., Urano, T., Suda, T., and Oike, Y. 2006. Angiopoietins and angiopoietin-like proteins in angiogenesis. Endothelium 13:71-79.
118. Murphy, E., Imahashi, K., and Steenbergen, C. 2005. Bcl-2 regulation of mitochondrial energetics. Trends Cardiovasc Med 15:283-290.
119. Harada, H., and Grant, S. 2003. Apoptosis regulators. Rev Clin Exp Hematol 7:117-138.
120. Guegan, C., Braudeau, J., Couriaud, C., Dietz, G. P., Lacombe, P., Bahr, M., Nosten-Bertrand, M., and Onteniente, B. 2006. PTD-XIAP protects against cerebral ischemia by anti-apoptotic and transcriptional regulatory mechanisms. Neurobiol Dis 22:177-186.
121. Vrana, K. E., Hipp, J. D., Goss, A. M., McCool, B. A., Riddle, D. R., Walker, S. J., Wettstein, P. J., Studer, L. P., Tabar, V., Cunniff, K., et al. 2003. Nonhuman primate parthenogenetic stem cells. Proc Natl Acad Sci USA 100 Suppl 1:11911-11916.
122. Vogel, G. 2005. Cell biology. Ready or not? Human ES cells head toward the clinic. Science 308:1534-1538.
123. Shibata, H., Ageyama, N., Tanaka, Y., Kishi, Y., Sasaki, K., Nakamura, S., Muramatsu, S., Hayashi, S., Kitano, Y., Terao, K., et al. 2006. Improved safety of hematopoietic transplantation with monkey embryonic stem cells in the allogeneic setting. Stem Cells 24:1450-1457.
124. Dufrane, D., Goebbels, R. M., Saliez, A., Guiot, Y., and Gianello, P. 2006. Six-month survival of microencapsulated pig islets and alginate biocompatibility in primates: proof of concept. Transplantation 81:1345-1353.
125. Jones, K. S., Sefton, M. V., and Gorczynski, R. M. 2004. In vivo recognition by the host adaptive immune system of microencapsulated xenogeneic cells. Transplantation 78:1454-1462.
126. Mai, G., Nguyen, T. H., Morel, P., Mei, J., Andres, A., Bosco, D., Baertschiger, R., Toso, C., Berney, T., Majno, P., et al. 2005. Treatment of fulminant liver failure by transplantation of microencapsulated primary or immortalized xenogeneic hepatocytes. Xenotransplantation 12:457-464.
127. Rahman, T. M., Diakanov, I., Selden, C., and Hodgson, H. 2005. Co-transplantation of encapsulated HepG2 and rat Sertoli cells improves outcome in a thioacetamide induced rat model of acute hepatic failure. Transpl Int 18:1001-1009.
128. Emerich, D. F., and Salzberg, H. C. 2001. Update on immunoisolation cell therapy for CNS diseases. Cell Transplant 10:3-24.
129. Levenberg, S. 2005. Engineering blood vessels from stem cells: recent advances and applications. Curr Opin Biotechnol 16:516-523.
130. Trounson, A. 2006. The production and directed differentiation of human embryonic stem cells. Endocr Rev 27:208-219.
131. Gerecht-Nir, S., Ziskind, A., Cohen, S., and Itskovitz-Eldor, J. 2003. Human embryonic stem cells as an in vitro model for human vascular development and the induction of vascular differentiation. Lab Invest 83:1811-1820.
132. Forbes, S. J., and Alison, M. R. 2006. Side population (SP) cells: taking center stage in regeneration and liver cancer? Hepatology 44:23-26.
133. Meissner, K., Heydrich, B., Jedlitschky, G., Meyer Zu Schwabedissen, H., Mosyagin, I., Dazert, P., Eckel, L., Vogelgesang, S., Warzok, R. W., Bohm, M., et al. 2006. The ATP-binding cassette transporter ABCG2 (BCRP), a marker for side population stem cells, is expressed in human heart. J Histochem Cytochem 54:215-221.
134. Asakura, A., and Rudnicki, M. A. 2002. Side population cells from diverse adult tissues are capable of in vitro hematopoietic differentiation. Exp Hematol 30:1339-1345.
135. Hierlihy, A. M., Seale, P., Lobe, C. G., Rudnicki, M. A., and Megeney, L. A. 2002. The post-natal heart contains a myocardial stem cell population. FEBS Lett 530:239-243.
136. Davidoff, A. M., Ng, C. Y., Brown, P., Leary, M. A., Spurbeck, W. W., Zhou, J., Horwitz, E., Vanin, E. F., and Nienhuis, A. W. 2001. Bone marrow-derived cells contribute to tumor neovasculature and, when modified to express an angiogenesis inhibitor, can restrict tumor growth in mice. Clin Cancer Res 7:2870-2879.
137. Urbich, C., Aicher, A., Heeschen, C., Dernbach, E., Hofmann, W. K., Zeiher, A. M., and Dimmeler, S. 2005. Soluble factors released by endothelial progenitor cells promote migration of endothelial cells and cardiac resident progenitor cells. J Mol Cell Cardiol 39:733-742.
138. Bug, G., Gul, H., Schwarz, K., Pfeifer, H., Kampfmann, M., Zheng, X., Beissert, T., Boehrer, S., Hoelzer, D., Ottmann, O. G., et al. 2005. Valproic acid stimulates proliferation and self-renewal of hematopoietic stem cells. Cancer Res 65:2537-2541.
139. Young, J. C., Wu, S., Hansteen, G., Du, C., Sambucetti, L., Remiszewski, S., O'Farrell, A. M., Hill, B., Lavau, C., and Murray, L. J. 2004. Inhibitors of histone deacetylases promote hematopoietic stem cell self-renewal. Cytotherapy 6:328-336.
140. Kijima, M., Yoshida, M., Sugita, K., Horinouchi, S., and Beppu, T. 1993. Trapoxin, an antitumor cyclic tetrapeptide, is an irreversible inhibitor of mammalian histone deacetylase. J Biol Chem 268:22429-22435.
141. Gagnon, J., Shaker, S., Primeau, M., Hurtubise, A., and Momparler, R. L. 2003. Interaction of 5-aza-2′-deoxycytidine and depsipeptide on antineoplastic activity and activation of 14-3-3sigma, E-cadherin and tissue inhibitor of metalloproteinase 3 expression in human breast carcinoma cells. Anticancer Drugs 14:193-202.
142. Fujieda, A., Katayama, N., Ohishi, K., Yamamura, K., Shibasaki, T., Sugimoto, Y., Miyata, E., Nishi, K., Masuya, M., Ueda, H., et al. 2005. A putative role for histone deacetylase in the differentiation of human erythroid cells. Int J Oncol 27:743-748.
143. Trowbridge, J. J., Xenocostas, A., Moon, R. T., and Bhatia, M. 2006. Glycogen synthase kinase-3 is an in vivo regulator of hematopoietic stem cell repopulation. Nat Med 12:89-98.
144. Dery, M. A., Michaud, M. D., and Richard, D. E. 2005. Hypoxia-inducible factor 1: regulation by hypoxic and non-hypoxic activators. Int J Biochem Cell Biol 37:535-540.
145. Laufs, U., Custodis, F., and Bohm, M. 2006. HMG-CoA reductase inhibitors in chronic heart failure: potential mechanisms of benefit and risk. Drugs 66:145-154.
146. Slangen, B. F., Out, I. C., Verkeste, C. M., Smits, J. F., and Peeters, L. L. 1997. Hemodynamic changes in pseudopregnancy in chronically instrumented conscious rats. Am J Physiol 272:H695-700.
147. Granger, J. P. 2002. Maternal and fetal adaptations during pregnancy: lessons in regulatory and integrative physiology. Am J Physiol Regul Integr Comp Physiol 283:R1289-1292.
148. Weiner, C. P., Knowles, R. G., and Moncada, S. 1994. Induction of nitric oxide synthases early in pregnancy. Am J Obstet Gynecol 171:838-843.
149. Trochu, J. N., Bouhour, J. B., Kaley, G., and Hintze, T. H. 2000. Role of endothelium-derived nitric oxide in the regulation of cardiac oxygen metabolism: implications in health and disease. Circ Res 87:1108-1117.
150. Iwakura, A., Shastry, S., Luedemann, C., Hamada, H., Kawamoto, A., Kishore, R., Zhu, Y., Qin, G., Silver, M., Thorne, T., et al. 2006. Estradiol enhances recovery after myocardial infarction by augmenting incorporation of bone marrow-derived endothelial progenitor cells into sites of ischemia-induced neovascularization via endothelial nitric oxide synthase-mediated activation of matrix metalloproteinase-9. Circulation 113:1605-1614.
151. Rubinstein, P., Dobrila, L., Rosenfield, R. E., Adamson, J. W., Migliaccio, G., Migliaccio, A. R., Taylor, P. E., and Stevens, C. E. 1995. Processing and cryopreservation of placental/umbilical cord blood for unrelated bone marrow reconstitution. Proc Natl Acad Sci USA 92:10119-10122.
152. Montuoro, A., De Rosa, L., Del Monte, C., Blandino, F., Pandolfi, A., Lanti, T., and De Laurenzi, A. 1991. A technique for isolation of bone marrow cells using hydroxyethyl starch (HES) sedimentation agent. Haematologica 76 Suppl 1:7-9.

What is claimed is:

1. A method of treating valvular heart disease comprising administration of cells, and/or products of said cells, and/or cell stimulatory compounds, and/or cardiac protective factors to an individual in need thereof.

2. The method of claim 1, wherein said cells are selected from a group consisting of: stem cells, committed progenitor cells, and differentiated cells.

3. The method of claim 1, further comprising administration of an antioxidant at a therapeutically sufficient concentration to a patient in need thereof.

4. The method of claim 3, wherein said antioxidant is selected from a group consisting of: ascorbic acid and derivatives thereof, alpha tocopherol and derivatives thereof, rutin, quercetin, allopurinol, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, retinoic acid, ACE Inhibitory Dipeptide Met-Tyr, recombinant superoxide dismutase, xenogenic superoxide dismutase, and superoxide dismutase.

5. The method of claim 1, further comprising administration of an antihypertensive, wherein said wherein said administration is prior to, and/or in combination with, and/or subsequent to cell therapy.

6. The method of claim 5, wherein said antihypertensive is chosen and administered in such a manner as to not substantially decrease coronary perfusion.

7. The method of claim 6, wherein said antihypertensive is a vasodilator.

8. The method of claim 7, wherein said vasodilator is selected from a group consisting of: hydralazine, cadralazine, felodipine, sildenafil, and nifedipine.

9. The method of claim 8, further comprising administration of an agent capable of causing afterload reduction, wherein said administration is prior to, and/or in combination with, and/or subsequent to cell therapy.

10. The method of claim 9, wherein said agent capable of causing afterload reduction comprises an angiotensin converting enzyme (ACE) inhibitor.

11. The method of claim 10, wherein said ACE-inhibitor is selected from a group consisting of: captopril, benazepril, enalapril, lisinopril, fosinopril, ramipril, perindopril, quinapril, moexipril, and trandolapril.

12. The method of claim 1, further comprising administration of an antibiotic agent capable of substantially inhibiting bacterial infections in the cardiac area, wherein said administration is prior to, and/or in combination with, and/or subsequently to cell therapy.

13. The method of claim 1, further comprising placing a cellular chemoattractant on, or in substantial proximity to the malfunctioning valve with the purpose of attracting therapeutic cell populations and activating said cell populations to regenerate valvular structures.

14. The method of claim 13, wherein said therapeutic cell populations are selected from a group consisting of: stem cells, committed progenitor cells, and differentiated cells.

15. The method of claim 13, wherein said chemoattractant is administered in the form of a depot injected intravalvularly, and wherein said depot is selected from a group consisting of: fibrin glue, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol.

16. The method of claim 13, wherein said chemoattractant is selected from a group consisting of: SDF-1, VEGF, RANTES, ENA-78, platelet derived factors, various isoforms thereof and small molecule agonists of VEGFR-1, VEGFR2, and CXCR4.

17. The method of claim 13, wherein said chemoattractant is administered into the area in need via transfection of a single or plurality of nucleotide(s) encoding said chemoattractant factor.

18. The method of claim 1, wherein prior to administration of cellular therapy, valvular repair is performed by surgical means.

19. The method of claim 18, wherein said surgical means comprises implantation of a prosthetic, or bioprosthetic graft, and wherein said cell administration is used in order to allow accelerated endothelialization, and/or allow prolonged efficacious function of said prosthetic, or bioprosthetic graft.

20. The method of claim 19, wherein said prosthetic or bioprosthetic graft is embedded with a chemoattractant of therapeutic cells in order to allow accelerated endothelialization, and/or allow prolonged efficacious function of said prosthetic, or bioprosthetic graft.

21. A method of preventing eccentric myocardial hypertrophy comprising administration of cells, and/or products of said cells, and/or cell stimulatory compounds, and/or cardiac protective factors to an individual in need thereof.

22. The method of claim 21, wherein said cells are selected from a group consisting of: stem cells, committed progenitor cells, and differentiated cells.

23. The method of claim 21, further comprising administration of an antioxidant at a therapeutically sufficient concentration to a patient in need thereof.

24. The method of claim 21, further comprising administration of an antihypertensive, wherein said wherein said administration is prior to, and/or in combination with, and/or subsequent to cell therapy.

25. The method of claim 24, wherein said antihypertensive is chosen and administered in such a manner as to not substantially decrease coronary perfusion.

26. The method of claim 21, wherein said antihypertensive is a vasodilator.

27. The methods of claim 21, further comprising administration of an angiotensin converting enzyme (ACE) inhibitor, wherein said administration is prior to, and/or in combination with, and/or subsequently to cell therapy.

28. The method of claim 21, further comprising administration of an antibiotic agent capable of substantially inhibiting bacterial infections in the cardiac area, wherein said administration is prior to, and/or in combination with, and/or subsequently to cell therapy.

29. The method of claim 21, further comprising placing a cellular chemoattractant on, or in substantial proximity to the malfunctioning valve with the purpose of attracting therapeutic cell populations and activating said cell populations to regenerate valvular structures.

30. The method of claim 29, wherein said therapeutic cell populations are selected from a group consisting of: stem cells, committed progenitor cells, and differentiated cells.

31. The method of claim 29, wherein said chemoattractant is administered in the form of a depot injected intravalvularly, and wherein said depot is selected from a group consisting of: fibrin glue, polymers of polyvinyl chloride, polylactic acid (PLA), poly-L-lactic acid (PLLA), poly-D-lactic acid (PDLA), polyglycolide, polyglycolic acid (PGA), polylactide-co-glycolide (PLGA), polydioxanone, polygluconate, polylactic acid-polyethylene oxide copolymers, polyethylene oxide, modified cellulose, collagen, polyhydroxybutyrate, polyhydroxpriopionic acid, polyphosphoester, poly(alpha-hydroxy acid), polycaprolactone, polycarbonates, polyamides, polyanhydrides, polyamino acids, polyorthoesters, polyacetals, polycyanoacrylates, degradable urethanes, aliphatic polyester polyacrylates, polymethacrylate, acyl substituted cellulose acetates, non-degradable polyurethanes, polystyrenes, polyvinyl flouride, polyvinyl imidazole, chlorosulphonated polyolifins, and polyvinyl alcohol.

32. The method of claim 29, wherein said chemoattractant is selected from a group consisting of: SDF-1, VEGF, RANTES, ENA-78, platelet derived factors, various isoforms thereof and small molecule agonists of VEGFR-1, VEGFR2, and CXCR4.

33. The method of claim 29, wherein said chemoattractant is administered into the area in need via transfection of a single or plurality of nucleotide(s) encoding said chemoattractant factor.

34. The method of claim 21, wherein prior to administration of cellular therapy, valvular repair is performed by surgical means.

35. The method of claim 21, wherein said surgical means comprises implantation of a prosthetic, or bioprosthetic graft, and wherein said cell administration is used in order to allow accelerated endothelialization, and/or allow prolonged efficacious function of said prosthetic, or bioprosthetic graft.

36. The method of claim 35, wherein said prosthetic or bioprosthetic graft is embedded with a chemoattractant of therapeutic cells in order to allow accelerated endothelialization, and/or allow prolonged efficacious function of said prosthetic, or bioprosthetic graft.

37. The method of claim 21, wherein said stem cells, and/or committed progenitors, and/or differentiated cells are administered into the left ventricle at a concentration sufficient to prevent, and/or reduce left ventricular diastolic end volume.

38. The method of claim 37, wherein said cells are administered locally into left ventricle myocardium.
